(19) United States
(12) Patent Application Publication
(10) Pub. No.: US 2023/0240991 A1
(43) Pub. Date: Aug. 3, 2023

(54) PROTEOPHOSPHOLIPOSOMES HAVING HDL-TYPE VESICLES

(71) Applicant: Ruth-Maria KORTH, München (DE)

(72) Inventor: Ruth-Maria KORTH, München (DE)

(73) Assignee: Ruth-Maria Korth, Munich (DE)

(21) Appl. No.: 17/300,210

(22) PCT Filed: Jul. 22, 2020
(86) PCT No.: PCT/DE2020/000163

§ 371 Date: Feb. 18, 2021

(30) Foreign Application Priority Data
Jul. 24, 2019 (DE) 20 2019 003 092.1
Nov. 8, 2019 (DE) 10 2019 007 769.5

(51) Int. Cl. A61K 9/127; A61K 38/38; A61K 47/12; A61K 31/07; A61K 31/375; A61K 31/593; A61K 31/355

(52) U.S. Cl. CPC A61K 9/1275; A61K 9/1276; A61K 38/38; A61K 47/12; A61K 31/07; A61K 31/375; A61K 31/593; A61K 31/355; A61K 9/1277; A61K 9/1271

(57) ABSTRACT

New proteophospholiposomes contain HDL-like vesicles with a new composite of anionic polypeptides, selected from the group of apoproteins A and at least one anionic polypeptide from the group of albumins, transthyretin-prealbumins and at least one cysteine group. The new anionic polypeptide composite is coated with layers of acyl-phosphatidylcholines that are protected from conversions by means of thio-phosphatidylcholines. The thiogroups attract antioxidants, ionic micromaterials and cofactors and are protected by exterior layers that contain neutral lipids and/or by means of capsules that contain microsomes, in an outwardly uniform fashion.

### TITLE OF PCT/DE2020/000163

Proteophospholiposomes with HDL-like vesicles containing a composite of apoproteins A with at least one polypeptide and at least one cysteine group and wherein the vesicles are surrounded with at least one layer containing acyl-phosphatidylcholines.

### 1. OBJECTIVE OF THE INVENTION

The novel proteophospholiposomes contain inner HDL-like vesicles with a composite comprising apoproteins A and polypeptides from the albumin group, the transthyretin-prealbumin group surrounded with at least one layer containing acyl-phosphatidylcholines which are stabilized with at least one thiolgroup. The thiolgroups bind ionic micromaterials and at least one cofactor, adapting special applications forms for protection of cells and/or for diagnostic use and for health promotion. The apoproteins A are selected out of the group comprising the albumin group, the serum albumin, lactalbumin, vitamin D-binding proteins and/or from the transthyretin-prealbumin group. The layers containing zwitterionic acyl phosphatidylcholines are stabilized with thio-phosphatidylcholines and are enriched for dietetic, dermal, transdermal, cosmetic, oral, nasopharyngeal, lacrimal, pulmonal, epimeningeal application forms and/or for diagnostic use. The dietetic compounds are prepared by preference with organic-certified materials comprising milk products, fish products, honey products, plant products, oily products. The proteophospholiposomes are prepared for special application forms. Ultrasound pulses and modified methods of ion exchange processes promote formation of vesicles (a), micelles (b), liposomes (c) and of multilamellar proteophospholiposomes (d) for dietetic, oral, pharyngeal, nasal, lacrimal, dermal, transdermal, cosmetic, pulmonal, cerebral, epimeningeal appliances. Diagnostic compounds and/or methods are prepared, performed with the modified ion exchange processes and/or with labeled thiolgroups, with labeled cysteine groups present in albumin to evaluate the capacity of the endothelium system. By preference, certified acyl-phosphatidylcholines are composed having esterified fatty acids, stearic acids, oleic acids, linoleic acids, linolenic acids (vitamin F). By preference organic-certified, semi-synthetic, synthetic sources are composed as starting materials are composed as ingredients and are prepared with biological extraction methods selecting by preference animal products, milk products, honey products and/or fish products especially fish oils, plankton products and/or plant products, especially seedlings or algae products. The cofactors are selected for special application forms by preference from the group comprising ionic micromaterials, acetyl coenzyme A and vitamins including retinol, cholecalciferoles, tocopherols. Ginkgoloides, xanthines can further promote then the netlike microcirculation of plexus systems in a cholinergic manner as cAMP-agonists. The proteophospholiposomes contain by preference an additional intermediate layer comprising albumin with cofactors as reservoir. The cofactors are selected for the skin by preference out of the group comprising glycoproteins, the filaggrins and the natural moistering factors. The pulmonal application forms are by preference enriched with synthetic dipalmitoyl-phosphatidylcholines as surfactant phospholipids and those are by preference synthetic, semisynthetic, purified proteophosphospholipids and are combined with steril liquids containing human albumin for bronchial installations. An isotonic buffer system is disclosed here as a model for pharyngeal, nasal, lacrimal, for inhalation routes, for epimengingeal dosage forms. Certified dipalmitoyl-phosphatidylcholines are prepared for pharyngeal, nasal, lacrimal, for inhalation routes, for epimeningeal dosage forms to protect cells and to stabilize endothelium systems and to promote the equilibration of plexus systems, of macrophages. At least one cysteine group activates here in the form of acetylcysteine thioclastic, xenobiotic systems against harmful peroxides, unfavorable materials especially during inflammatory disorders. The application forms are also used to promote the lymphomeningeal drainage systems of the bidirectional transport systems and especially of the Choridoid Plexus using the epimeningeal, lacrimal, nasolacrimal, nasopharyngeal, pharyngeal, buccal, lingual, dermal, parenteral dosage forms. The inner HDL-like vesicles protect cells with the novel polypeptide composite containing apoproteinsA, transthyretin-prealbumin, cysteine groups which are surrounded with certified semisynthetic, synthetic dipalmitoyl-phosphatidylcholines, wherein cofactors such as apoproteinsE2 and the reelin system activate the VLDL-related systems. The cholinergic proteophospholiposomes promote cells, the uptake of nutritionals and the thioclastic system. Transthyretin-prealbumin is added here to inhibit re-absorption of harmful albumin adducts. For diagnostic procedures spin label markers are added by preference with cysteine-thiolgroups to detect oxidized, transformed carrier proteins and for protection of cells and to determine the intestinal energy flow without irritations. The labeled proteophospholiposomes are suitable for imaging procedures.

### 2. SUMMARY OF THE INVENTION

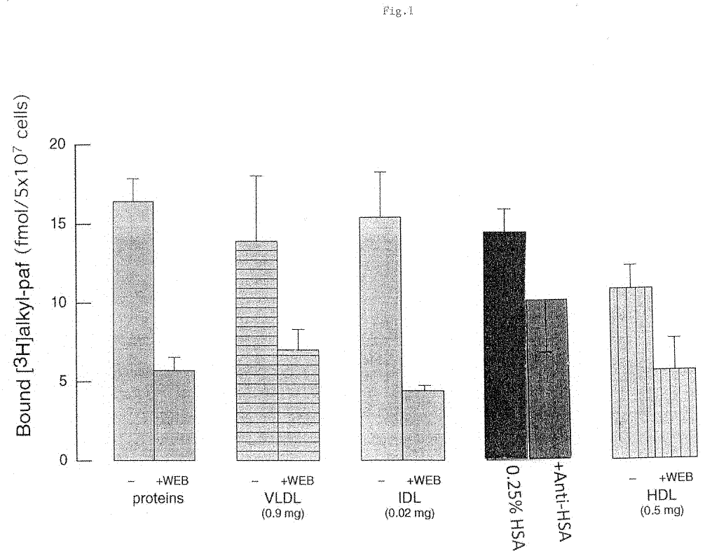

Apoproteins A are composed for the first time with at least one peptide from the albumin-group and/or the transthyretin-prealbumingroup and those are linked with cysteine groups. The novel composite containing anionic polypeptides bind zwitterionic acyl-phosphatidylcholines in the form of inner HDL-like vesicles, coated by one or more layers containing acyl-phosphatidylcholines, which are stabilized with enriched thiogroups, with cysteine groups as well. Moreover, at least one layer contains albumin enriched with cysteine-residues, ionic micromaterials and vitamins having a broad buffer capacity and those layers are surrounded with one or two exterior lipid bilayers. The inner HDL-like vesicles protect here human cells of the whole body. The starting materials are milk products and/or plant products such as nuts, seedlings serving as materials containing methionine-cysteine and acyl-phosphatidylcholines, acetyl coenzyme A and cAMP-agonists as well. Unpublished experiments provide a practical model and show protection of intact cells in response to HDL-proteins and in the presence of an isotonic, enriched buffer solution, suitable as carrier system for pulmonal, parenteral, subcutaneous, epimengingeal, direct, topical installations (FIG. 1). These dispersions contain at least 0.5 mg/ml apoproteins A with 0.25% delipidated serum albumin and at least 11.9 mM Na HCO3; 137 mM NaCl, 2.68 mM KCl, 1 mM MgCl2, 0.41 mM NaH2PO4; 0.5 mM dextrose. The HEPES-sulfate group (5 mM) is replaced by cysteine (30 mg/100 ml) and acyl-phosphatidylcholine are added (um 500 nM). The formation of HDL-like vesicles (a) is enhanced with ultrasound pulses (4° C.), whereby the vesicles are enriched with cationic surface materials by means of cationic reflections forming themselves as micelles (b) in a repeated oil in water situation and the micelles are enriched using reflections by anionic surface materials and then liposomes (c) form themselves in an external neutral lipid medium and form Proteophospholiposomes (d) which are dissolved in eluates having appropriate charges. The vesicles (a), the micelles (b), the liposomes (c), the multilamellar proteophospholiposomes (d) are prepared and/or enriched alone and/or together whereby eluates with appropriate charges prepare the particles in an outwardly uniform fashion for cosmetic, dermal, dietetic products with capsules, with micro-liposomes.

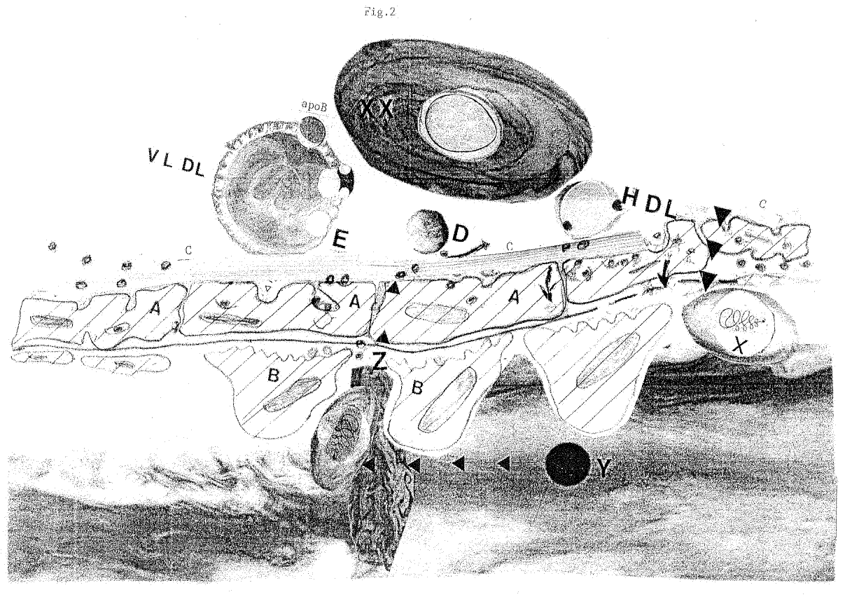

The synergistic defense systems are disclosed here for the first time counteracting VLDL-related defects of cells (Tables 1+2). The protection system comprises functional HDL-influx capacity, normal levels of albumin and robust endothelium barriers antagonizing VLDL-related cellular defects and disturbed influx-efflux-systems of cholesterol (Tables 1+2). Quite surprisingly, elevated levels of plasma albumin overlap with a relevant vasopermeability and with diastolic hypertension even among abstinent persons having otherwise normal values. Thus, a dual effect of albumin is disclosed for the first time indicating here pH-dependent problems of the perivascular system. Moreover, an increased vasopermeability was observed among elderly persons and was distinguished from enhanced vasopermeability during chronic alcohol consumption using novel FiDA®-algorithms (Tables 1+2). The alcohol-related problems show here clear progression starting from albuminuria and then with diastolic hypertension, a disturbed cAMP-related vasodilation of vascular smooth muscle cells. The proteophospholiposomes counteract increase levels of urinary albumin by means of cell protection as the composite contains apoproteins A (namely with apoproteins A1, A2) and at least one albumin from the group of plasmatic albuminoid transporters and/or the transthyretin-prealbumin group enriched with cysteine groups. The cysteine groups stabilize the proteophospholiposomes, interact with heparan sulfates of the anionic, luminal matrix and attract ionic micromaterials. The interaction is mediated by the proteophospholiposomes starting from a stationary medium during the preparation process which alternately contain peptides and/or fatty components. The endogenous absorption of the proteophospholiposomes is enhanced by means of exterior neutrally charged layers. The interstitial pH-values remain here robust with equilibrated proportions of lipids (F) and proteins (E) (FEQ: e.g. 1.4/1, vol/vol). The intercellular passages are promoted without irritations of the entire endothelium barriers (FIG. 2). The applications equilibrate the cholinergic systems especially the network of neurovascular plexus systems. The proteophospholiposomes are the delivery systems then of nutritional factors as a reservoir, as albuminoid transporters of ionic micromaterials to equilibrate the energy flow. Thus, the novel multilamellar proteophospholiposomes are prepared, buffered, titrated for special, unexpected applications and dosage forms to protect cells of sensitive and/or elderly persons who tend to oxidative stress and/or for persons having a diminished energy capacity.

Own unpublished data disclose here for the first time that purified HDL-like proteins protect the membranes of washed intact human cells (FIG. 1). Experiments show here for the first time that HDL-proteins protect intact human cells against alcohol metabolites from the group comprising alkyl-phosphocholines (alkyl-GPC, LA-PAF). A relevant embodiment of the invention comprises the preparation of/as a novel unexpected composite containing apoproteins Aa, A2, E2 (here apoproteins A) with at least one peptide from the group comprising albumin-prealbumin. The anionic polypeptides contain cysteine groups and are lipidized, protected, surrounded, buffered, titrated with one or more layers containing acyl phosphatidylcholines. The background art concerning human membranes serves as a general model. Membranes from intact human cells are protected here with the proteophospholiposomes stabilized with cysteine groups as those are vulnerable containing vulnerable phosphatidylcholine layers as well.

A further embodiment of the invention refers to the novel preparations of/as proteophospholiposomes for special application forms and for a cholinergic equilibration. Application forms are developed considering neurovascular networks which are concentrated e.g. in plexus systems. The proteophospholiposomes mediate protection of cells in the presence of apolipoproteins A in a composite with albumin, with transthyretin-prealbumin whereby cytidine phosphates, acetyl coenzyme A additionally promote the cholinergic effects of choline phosphates in the presence of vitamins from the B-group. The unexpected proteophospholiposomes bind thio groups and ionic micromaterials promoting therewith the energy flow. The physiological pH values are adapted here considering the pH values of compartments of interest using buffer systems as shown with experiments. The unexpected protection of human cells by HDL-proteins is shown here for the first time in the presence of delipidated albumin dissolved in a sulfate-phosphate buffer. Thiogroups in the proteophospholiposomes enrich ionic micromaterial, promote ionic pulsations of interstitial spaces and activate the xenobiotic gluthation system for the thioclastic excretion of unfavorable, of harmful materials. The cysteine groups of the proteophospholiposomes mediate disulfide bonds, tend to sulfit exchange including interactions with heparan sulfates of matrix materials and increase endogenous absorption as e.g. exterior layers of neutrally charged lipids modulate an anion reflections. The novel proteophospholiposomes are covered with neutral lipids and comprise inwards directed layers containing zwitterionic acyl-phosphatidylcholines (80%). Intermediate layers contain polypeptides and/or glycoproteins as cofactors. These multilamellar proteophospholiposomes are prepared, buffered, titrated and are adapted with one or more lipid bilayers for specific application forms and to endogenously protect cells. The preparation uses electronical charges and capacities of the above mentioned configurated polypeptides which are composites with cysteine groups forming a novel polypeptide. The albumin can be selected for/as preparation from the group comprising whey proteins (lactalbumin, lact-globulins) and/or the transthyretin-prealbumin group as hormone carrier. The novel proteophospholiposomes are based on the unexpected synergistic relationship of human serum albumin with cAMP-dependent influx capacity of purified HDL-peptides which are enriched here with ionic micromaterials, cofactors, antioxidants in the experiments. The stationary media are also buffered and titrated and can be used as carriers of the desired particles. The endogenous pH values are used as corresponding presentation for titrations to enhance the perfusion and the endogenous influx-efflux systems. The anionic matrix materials and the pH values of the interstitial systems are considered here for the first time with preparations of liposomes in general. The entire endothelium system is protected considering the netlike structures e.g. of the plexus systems and that regeneration is possible. The luminal, intercellular, subendothelial structures are protected to equilibrate increased levels of plasma albumin as well and to antagonize an enhanced emigration of albumin disturbing here the cAMP-dependent vasodilation of vascular smooth muscle cells in a clinically relevant manner. In addition, the size of the proteophospholiposomes is adapted to the nanometer size of endothelial pores and of the endogenous intercellular spaces. Redox cycles are substituted with vitamins A, B, C, D, E like certified organic milk products as contemplate.

As further embodiment of the invention, the application forms and the proteophospholiposomes are prepared considering the novel FiDA® Algorithms. Critical FiDA® Algorithms show here for the first time relevant deficits of the HDL-related protection capacity and unveil alcohol problems in an objective manner. The protection systems comprise here the interaction of albumin with the HDL-mediated cell protection (Tables 1+2). The novel FiDA® algorithms distinguish relevant alcoholic risk profiles from sporadic problems so as an objective evaluation of risk profiles is possible.

A further embodiment of the present invention comprises the enrichment of labeled or unlabeled components for diagnostic tests and testing procedures, for in vitro tests and/or for imaging processes. The thiogroups and/or the C34 cysteine groups of albumin are labeled by preference with spin-label-markers and endogenous passages are then followed using the labeled proteophospholiposomes. The labeled proteophospholiposomes pass barriers and are then endogenously reduced layer by layer as those are prepared layer by layer. The multilamellar proteophopsholiposomes are formed, enriched, buffered, titrated, purified layer by layer using by preference the ion exchange processing of the inner priority document PA10 2019 007 769.5 protecting a novel test and a testing procedure for quantitative and qualitative measurements of the ligands and/or of the proteophospholipids. The energy flow of the interstitial systems is then not disturbed during the diagnostic procedures. The labeled proteophospholiposomes are especially suitable for electromagnetic testing procedures of entire endothelium systems because the endothelium barriers are not irritated then. The entire endothelium barriers further comprise the endogenous matrix, the luminal, intercellular, subendothelial structures especially of the fenestrated endothelium barriers and/or pores of confluent endothelium systems (FIG. 2). The novel proteophospholiposomes endogenously protect especially membranes and cells also of sensitive or elderly persons who often tend to an increased oxidative stress and tend to reduced energy. The novel proteophospholiposomes are especially suitable for spin-label markers to evaluate the quality and thickness of matrix materials and of the basement membranes. Especially important is a rapid diagnostic processing suspecting acquired fenestration of capillaries indicating an enhanced angiogenesis e.g. during tumors or during diabetes, and to avoid then dramatic aggravations e.g. during pneumonias.

Overall, the novel proteophospholiposomes are prepared for protection of membranes and cells, for equilibration of the energy flow, for the ATP-dependent influx-efflux-systems as the efficacy and regeneration of these systems are decreased during aging. The novel proteophospholiposomes are also prepared for sensitive persons, for children, for pregnant women and against nutritional problems and also for psycho-vegetative equilibration during psycho-vegetative stress. The proteophospholiposomes are especially suitable for abstinent persons with alcohol problems as the proteophospholiposomes are prepared without fatty alcohols. Certified organic components are used as starting materials which can be completed with semi-synthetic products by preference for non-invasive application forms. Synthetic components are by preference used for invasive application forms, for example with synthetic dipalmitoyl-phosphatidylcholines for direct installations of surfactant phospholipids.

### 3. STATE OF THE ART AND BACKGROUND OF THE PRESENT INVENTION

Applicant's prepublished intellectual property rights disclose prescriptions with oily Ginkgoloides enriched with cholecalciferoles (vitamin D) and/or with acetylcysteine wherein commercially available liposomes are used but those have not been titrated before. In Applicant's prepublished U.S. Pat. No. 10,517,383B2 (publ. Feb. 27, 2011) the Ginkgoloides are disclosed as group comprising PAF antagonists inhibiting the receptors of alkyl-acetyl-sn-glycero-phosphocholines (PAF, U.S. Pat. No. 10,517,383B2) and those can antagonize lipidation of cells. LDL and/or purified ApoB are the risk factors enhancing then the harmful accumulation of alkyl-phosphocholines in human cells. The classical preparation of plasma fractions by ultracentrifugation and dialysis is also prepublished (e.g. R. Korth et al., Chem. Phys. Lipids, Vol. 70, 1994). The Ginkgoloides were selected as competitive inhibitors of affine receptors interacting with chemically defined 1-O-alkly-acyl-sn-glycero-3-phosphocholines (LA-PAF, alkyl-GPC) prior to the invention. The Ginkgoloides were selected before from the group comprising triazolothienodiazepines (Boehringer. Germany), the paf analogues (Takeda, Japan), the Ginkgolides (Beaufour, France) using the previously discovered novel acetyl receptors interacting with the acetyl groups without allosteric upregulations in response to alkyl-GPC. Furthermore, Applicant showed with another patent prescription (EP 2599391, Publ. Jun. 6, 2013) new biological extraction processings to obtain pure lecithins, chemically acyl-acyl-glycero-phosphatidylcholines, whereby C16-lecithins further extract C18-lecithins. The preparation of lecithins of and with lecithins starts with membranes extracted with synthetic C16-phosphatidylcholines whereby increasingly lipophilic lecithins are enriched as fatty oils, as plant butter allowing uptake of fatty Ginkgolides. Prepublished prescriptions of and with certified organic lecithin oils were shown in advance containing by preference omega-3-fatty acids suitable for sensitive persons suffering of overweight, hyperlipidemia, intolerance to glucose and/or acne and intestine problems. Applicant's prepublished intellectual property rights provide many own screening methods using washed human cells.

Furthermore, it is known that albumin originates from hepatic präpro-proteins (about 12 g albumin per day) and is then enriched as major transport protein of minerals and antioxidants in the plasma. Albumin contains polypeptide chains comprising 585 amino acids and 17 disulfide bridges stabilizing configurations and form pockets for ligands which are then protected against oxidation. Albumin is a reservoir, a delivery systems, an acceptor also of free fatty acids, of hormones. Sterile albumin products are commercially available. Apoproteins A1A2 from human plasma are commercially available and also recombinant proteins from cells in culture (e.g. wwww.sigma-aldrich.com). Dipalmitoyl-phosphatidylcholines, cysteine, N-acetyl-cysteine, acetyl Coenzyme A, vitamins, minerals, spin label markers of the thiol groups, from albumin are commercially available as well. Thio-phosphatidylcholines (Boehringer) were tested in advance as complementary medicine during uptake of cytostatica (W. Berdel, R. Korth et al. Anticancer Research, Vol. 7, 1987).

Commercially available liposome kits contain phosphatidylcholines (PC), cholesterol (C) and stearic amines (7:2:02). The commercially available liposomes are characterized as “lipid bilayers”, as bilayers defined by lipids which can trigger local irritations. Moreover, the commercially available liposomes were neither buffered nor titrated and can then promote formation of aggregates, of precipitations and especially oxidation of lipids. The commercially available and/or overlipidated liposomes cannot teach or render obvious the present invention. According to the invention, the liposomes are titrated here for the first time caring about the lipid-protein quotient (FEQ). Elevated lipid to protein ratios (FEQ) trigger ketosis and decreased levels of the ratio trigger acidosis which are avoided here to promote the endogenous perfusion. The novel proteophospholiposomes comprise by preference an equilibrated FEQ-ratio wherein the summarized proportions of lipids/phospholipids to summarized protein avoid an endogenous change of pH values. An equilibrated lipid-protein quotient is reached here with an FEQ of about 1.3. For example, the lipid components of enriched milk products are about 4% and the protein proportions are about 3% in final product. Said lipid-protein quotient (FEQ) is equilibrated here for the first time in the unexpected proteophospholiposomes of the present invention.

### 4. TECHNICAL PROGRESS OF THE PRESENT INVENTION

An important technical progress discloses the novel composite containing polypeptides and cysteine groups wherein HDL-like vesicles are coated for the first time with certified organic acyl-phosphatidylcholines (80%) and those are stabilized with thio-phosphatidylcholines for the first time. A novel composite containing apolipoproteins A and albuminoids is coated with protective acyl-phosphatidylcholines. These unexpected proteophospholiposomes are enriched, buffered, titrated with novel preparation methods. An important technical progress discloses that a novel composite contain protective polypeptides. For the first time, apoproteins A are conjugated by means of cysteine groups which are present in the peptides or which are enriched therein. The novel anionic HDL-like composite consisting of peptides (pH about 6.5) binds at least one layer of zwitterionic acyl-phosphatidylcholines (pH about 7.4), wherein the polypeptides and the phospholipid layers fusion by means of cysteine groups arranging near distances in stationary media which are buffered, titrated in an appropriate manner. The stationary media can be used as carriers or the vehicles are separately prepared and taken by carriers which are not limited. The inner HDL-like vesicles (a) contain the anionic composite consisting of polypeptides and arrange themselves with the zwitterionic acyl-phosphatidylcholines. The anionic polypeptides are selected out of the group comprising apoproteins A, the albumin group and the transthyretin-prealbumin group which are suitable for conjugations by means of cysteine groups. The proteophospholiposomes are prepared layer by layer whereby only certified components are prepared.

Thiogroups and thiobonds are used here for the first time for stabilization of acyl-phosphatidylcholines (80%) with thioether-phosphatidylcholines by preference with 1-acyl-2-thioether-phosphatidylcholines. Especially the thiobonds in the position 2 are not sensitive to hydrolysis by lipases, phospholipases, acetylhydrolases. Moreover, thiogroups activate cytidine-phosphates, thioclastic xenobiotic systems and attract ionic micromaterials promoting the energy flow of endogenous, interstitial spaces. The proteophospholiposomes are prepared layer by layer and are protected for the first time against oxidation, hydrolysis and/or against endogenous transformations. The pH values of the local microsystems are especially sensitive during dermal, transdermal, dietetic appliances reaching the cerebral flow systems e.g. upon passages of the Plexus Pharyngeus. The thiogroups require equilibration of redox cycles. Layer by layer is buffered here whereby also the stationary media are enriched with the vitamins A, B, C, D, E, F and/or with natrium-hydrogen carbonates in stationary media, in oily or watery media. The broad antioxidant buffer capacity of albumin is used (0.25% BSA of 0.25% HSA). The carrier systems are also buffered in a full and appropriate manner. The proteophospholiposomes contain then the HDL-like inner vesicles and further contain cysteines, antioxidants and by preference certified organic diacyl-phosphatidylcholines, wherein the covering layers are also stabilized with thiogroups stabilizing with electron clouds the binding of ionic micromaterials and cofactors.

An important technical progress discloses the novel cysteine-containing polypeptides which widely arrange themselves as “multi-unit-peptides”. The unexpected HDL-like peptide group is developed here as novel “inner HDL-like vesicles” which are further lipidized, protected, coated with one or more layers containing acyl-phosphatidylcholines. By preference certified acyl-phosphatidylcholines (80%) are prepared as layers stabilized with thiogroups. The lamellar preparation is reached by alternative incubations in oily and aqueous media which are enriched, buffered, titrated as well. Neither fatty alcohols, nor emulsifiers, nor detergents are added during the preparation processes.

An important inventive step discloses the conditions of electrostatic capacities, wherein anionic peptides attract zwitterionic acyl-phosphatidylcholines which are stabilized here also with thiogroups. The thiogroups form biologically active cytidine phosphates and acetyl Coenzymes A which trigger together with choline groups, with choline phosphates the (re)synthesis of acetylcholines to equilibrate disturbed influx-efflux systems which are verified here for the first with the novel FiDA® formula in an objective manner (Tables 1+2). The novel proteophospholiposomes are enriched with cAMP-agonists, e.g. with cholecalciferoles to protect against said problems and to promote favorable cAMP-dependent functions especially of sensitive persons, of children, pregnant women, of elderly persons and/or during alcohol problems (Tables 1+2). The essential methionine- and cysteine-groups are used as starting materials forming then thiobonds connecting symmetrical, asymmetrical, neutral charges and/or of phospholipid layers with polypeptides. The vulnerable acyl-phosphatidylcholines are stabilized here for the first time with thioester-, thioether phosphatidylcholines wherein their very reactive electron clouds activate thioclastic systems. The thio-exchange reactions attract ionic micromaterials and require substitution of redox cycles.

The protection of cells with HDL-proteins is shown here with own unpublished data for the first time (Tables 1+2). Intact human thrombocytes are protected here for the first time with HDL-proteins in a buffered, titrated model system and in the presence of purified serum albumin (FIG. 1). The cerebral cells are protected as well against fatty alcohols by preference with an intermediate of meningeal drainage systems. The known “remodeling pathways” of the brain can transform physiological plasmalogenes into damaging alkyl-acyl-glycero-phosphocholines disclosing here for the first time cleavage by means of albumin with yet unpublished statistical estimates of cerebrospinal fluids (CSF) representing meningeal drainage systems (CSF).

A preferred embodiment of the invention disclose the combination of pure, purified and/or synthetic polypeptides with acyl-phosphatidylcholines. Apoproteins A and albumin are commercially available and/or are purified and prepared by preference with the modified ion exchange procedures of the priority document PA102019007769.5. Facile, practicable, dietary preparations are composed here with commercially available apoproteins A and with delipidated albumin (1/1, Vol/Vol) and are then lipidized and enriched with certified organic milk products containing cysteine (30 mg/100 ml). Certified organic acyl-phosphatidylcholines are selected here containing esterified linoleic acids, linolenic acids (about, 105 mg/100 g) and are enriched with omega-3-fatty acids. The novel proteophospholiposomes are stabilized then adding cysteines which stabilize the novel polypeptide composite and the zwitterionic acyl-phosphatidylcholines. The phospholipid layers can be further composed with cationic phosphatidyl-ethanolamines to promote lipid bilayers (bilayers) which arrange themselves.

A further embodiment of the invention discloses the implementation of the unexpected synergistic interaction of albumin and HDL which is clinically realized here for the first time (Tables 1+2) with an increased vasopermeability. A protection of cells is reached as example with representative HDL-proteins, namely with apoproteins A and indeed in the presence of delipidated albumin with yet unpublished experiments. Fat free plasma proteins alone fail protection of intact thrombocytes against fatty alcohols and VLDL-proteins (apoproteins B+E) and fail protection of cells against the ether groups of fatty alcohols which are tested here with alkyl-acetyl-glycero-phosphocholines (FIG. 1). Certified diacyl-phosphatidylcholines of the proteophospholipids have been selected before using tests so as no ether linkages are included during the preparations procedures. The major proportion of healthy human membranes contains diacyl-phosphatidylcholines (80%). The vulnerable ester bonds are stabilized here with thiogroups to avoid defects of cells in response to fatty alcohols. The thiogroups stabilize acyl-phosphatidylcholines also against oxidation and against degrading enzymes and thiogroups require full redox cycles. In accordance with the invention, the cleavage of harmful materials is enhanced by means of enriched albumin transporters containing by preference acetylcysteines and/or cytidine phosphates.

A further embodiment of the invention discloses for the first time that transthyretin-prealbumin is fusioned with apoproteins A adding cysteine and the composite is then lipidized with acyl-phosphatidylcholines and is stabilized with thiogroups and minerals such as e.g. silicates. Moreover, one or more intermediate layers are prepared here containing by preference enriched albumin-adducts which can further bind selected cofactors. The cofactors are selected from the group comprising endogenous helper factors, by preference the glycoproteins, the apoproteins E, the reelin systems, the cAMP agonists, the natural moisturizing factors of the skin. The endogenous influx-efflux systems are promoted, for example, with apoproteins E2, with cAMP-stimulating xanthines, with filaggrins promoting the strength of the endothelial barriers and/or the energy flow by means of acetyl coenzymes A and/or with heparan sulfates for interstitial functions. The reelin peptides represent a composite capable of activating the cerebral efflux systems by means especially of functional, subendothelial receptors interacting with VLDL and apoproteins E and those are linked with functional ATP-dependent transmembrane transport proteins. In accordance with the invention, the interstitial energy flow is promoted as especially thiogroups attract micromaterials e.g. silicates. Moreover, the symmetrically or asymmetrically charged phospholipids further bind the vitamins A, B, C, D, E, F equilibrating redox cycles.

As a further embodiment of the invention, adapted dosage forms are disclosed promoting plexus systems in a balancing manner. The cysteine groups form cytidine phosphates and choline phosphates forming then acetyl coenzymes A in the presence of the vitamin B group promoting then cholinergic systems of Plexus systems and (re-)synthesis of acetylcholines. The cholinergic compounds promote the neuro vegetative equilibration reaching especially netlike vessels of the hypodermis, of the plexus systems. For example, the drainage systems of Plexus Abdominalis, Pulmonalis, Pharyngeus are targets and/or are connected with intermediate of the Sinus Cavernosus with the Chorioid Plexus and/or with the lymphatic and/or meningeal drainage systems. In addition, cholinergic compounds protect cells, balance macrophages and are effective as tissue hormones for vasodilation. Acetylcholines diffuse together with micromaterials into perivascular spaces. The macromaterials pass cellular membranes via ATP-dependent specific transport systems. The endogenous release of micromaterials such as acetylcholines dilate vessels, enhance fluidity of interstitial gels and the ionic pulsations so as the blood flow and the perivascular, perineuronal systems are promoted. Furthermore, thiogroups further interact with heparan sulfates of the matrix and promote the energy flow by means of acetyl Coenzyme A enhancing the cAMP-dependent regulatory response elements of blood pressure, of the HDL-dependent influx systems and of the hepatic gluconeogenese. The proteophospholiposomes equilibrate netlike plexus systems, deliver nourishing compounds, nutritionals, supplements and activate xenobiotic cleavage systems enhancing excretion of useless metabolites, of precipitates, of aggregates, of cellular rest materials, of amyloids which could e.g. accumulate in more relevant amounts, for example during inflammations or cytostatic therapy.

A further unexpected embodiment of the invention discloses new, labeled proteophospholiposomes for diagnostic appliances. The procedures of the priority document PA 1020 19 00 7769.5 are included by citation. In accordance with the invention, the proteophospholiposomes are labeled for diagnostic appliances whereby commercially available spin-label-markers are used such as e.g. labeled thiolgroups and/or labeled cysteine groups which are present in albumin but not limited to albumin. As a special embodiment of the invention, the novel proteophospholiposomes are provided with labeled compounds for diagnostic procedure and can be measured by preference with electrical, electromagnetic, spin-labeled technical methods. The diagnostic appliances can be modified for diagnostic preparations, for procedures in vitro, in vivo and/or for imaging procedures (e.g. EBR, NMR). Electrical charges are used also in the priority documents for devices, preparations, purification methods. The preparation of proteophospholiposomes comprises the unexpected ion exchange processings of the priority document as well. The novel proteophospholiposomes can be labeled with electrical, magnetic markers which are suitable as contrast media, for imaging procedures. Especially the below mentioned evaluation of the clinical Tables demonstrates that said tests and testing procedures are required for an evaluation of emigrated albumin by preference using non-acidified urine samples and/or for continued diagnostic procedures of albumin in the lymphomeningeal drainage systems. The specified tests of the priority documents are included by citation using by preference pH-dependent matrix-like surfaces improving the qualitative and quantitative testing of emigrated albumin. In accordance with the invention, a relevant vasopermeability is verified here with means of the critical FiDA® formula and/or during hepatorenal syndromes which are combined here with diastolic hypertension and require further diagnostic procedures (Table 1+2).

A further embodiment of the invention discloses the enrichment of phospholipid layers to form bilayers as interior membranes arranging themselves by means of zwitterionic phosphatidylcholines (80%) and phosphatidylethanolamine (<10%). These cationic layers further bind at least one anionic peptide layer. The vesicles are then transformed into micelles in a watery medium having then exterior anionic layers (pH6.5) which can then attract again neutrally charged lipids (about pH7.4). By preference neutrally charged lipids form exterior layers and those bind lipophilic cofactors such as retinol (vitamin A), cholecalciferoles (vitamin D), xanthines as cacao butter and/or with Ginkgolides as Ginkgolide butter. Especially the lipophilic retinol promotes absorption due to specific transporters overlapping with transmembrane binding sites of the transthyretin group. The proteophospholiposomes are coated by preference with fatty materials and/or with capsules for a nanomolar release of the components. Proteophospholiposomes contain here albumin as reservoir and albumin should be protected as well e.g. with capsules against intestinal degradation implicating oral, intestinal uptake. The proteophospholiposomes are surrounded here with protective coverings to reach neuro vegetative networks of plexus systems for the neuro vegetative balance, for the energetic equilibration of cholinergic systems.

In a further embodiment of the invention, the proteophospholiposomes are prepared, stabilized by preference to reach plexus targets because the plexus systems connect capillaries, venous systems with the lymphatic and neuro vascular systems. The entire endothelium systems comprise here the luminal matrix materials, the endothelium layers and the subendothelial systems (FIG. 2). The endogenous pH values of these systems are considered here for promotion of unidirectional lymphatic vectors. Ionic interstitial pulsations can be used for diagnostic appliances by means of labeling or the electromagnetic capacities are enhanced by means of ionic micromaterials. The ionic micromaterials diffuse and improve the fluidity of interstitial systems. Barriers, cells, cellular membranes are protected here for the first time with a novel composite containing HDL-proteins coated with acyl-phosphatidylcholines and with thiogroups which form cytidine phosphates and choline phosphates favoring reactions and equilibrate cholinergic neuro vascular structures and further promote thioclastic, xenobiotic drainage systems. The direct protection of cells is disclosed here for the first time with own unpublished experiments as specified below. Indeed, apoproteins A protect intact, washed human thrombocytes in the presence of delipidated albumin and in a HEPES phosphate buffer containing sulfate groups as shown below. Moreover, an improved diagnostic is disclosed in the priority document PA 10 2019 007 769.5 using electrostatic capacities for the first time for novel test devices comprising electromagnetic fusion partners and/or unexpected preparations methods for manufacturing and for purification of proteophospholipids and those are included here by citation. In accordance with the invention, the novel proteophospholiposomes are adapted to antagonize the unexpected influx-efflux problems which overlap with fatty alcohols and/or with elevated vasopermeability of elderly persons. The novel proteophospholiposomes are applied here for protection of cells and to repair influx-efflux problems which are related with HDL (Tables 1+2, FIG. 1).

### 5. PREPARATIONS OF AND WITH PROTEOPHOSPHOLIPOSOMES

An important embodiment of the invention discloses preparations starting with dispersions (FIG. 1). Albuminoid transporters are brought near to each other which are selected from the group comprising apoproteins A1, A2, E2, the albumin group and/or the transthyretin-prealbumin group and which can be further titrated then in an anionic manner with acetylcysteines. The anionic polypeptide adduct arranges itself in watery media (≥20° C.) and is then lipidized by means of oily media containing zwitterionic acyl-phosphatidylcholines, esterified by preference with palmitoyl, stearic, oleic acids, with linoleic, with linolenic acids and are stabilized with cysteine groups and antioxidants. The aqueous stationary media are enriched with peptides, with ionic micromaterials, with antioxidants, with cofactors as well so as lamellar vesicles, micelles, liposomes arrange themselves by means of an optional change of watery or oily media. The technical procedures, enrichments and purifications are performed at cool temperatures (e.g. ultrasound pulses, ionic exchange procedures, centrifugations). The incubation with alternately changed media can be repeated several times until the desired size is reached of the multilamellar proteophospholiposomes. The stationary media are provided during the preparation only with sterile, buffered, certified components to avoid formation of fatty alcohols, of ether lipids, of ether phospholipids which be excluded using selection methods. For example, an anionic dispersion of peptides is prepared in an initial medium and is then connected with an oily medium. Cold ultrasound pulses promote then formation of vesicles (4° C.). The vesicles have cationic surfaces (a) or neutrally charged surfaces of the proteophospholiposomes (d) enabling accumulations, purifications by means of reflections using outsourced e.g. cationic surfaces whereby oily eluates can take the oily proteophospholipids e.g. as anionic oils. The modified ion exchange procedures of the priority documents are included here by citation and are specified below. The procedures can be repeated several times to form multilamellar liposomes which are enriched, titrated, buffered layer by layer. The phospholipid layers can be titrated in a cationic manner with phosphatidylethanolamines to bind intermediate layers containing anionic peptides which are enriched by means of ionic exchange procedures as well based on the priority documents and/or which can be purified maintaining a favorable lipid(fat)-protein-quotient (FEQ) of the proteophospholiposomes as a whole if possible. The experimental buffer system serves a representative model because HDL-proteins protect here intact cells against fatty alcohols for the first time as shown with own unpublished experiments (FIG. 1). The novel proteophospholiposomes are prepared here with eco-certified components and are buffered, titrated for special application forms wherein the carriers are also buffered and titrated for regeneration compounds and/or for diagnostic procedures of neuro vascular systems without irritation of endothelial systems.

An important option of the invention is that preparation (A) can be easily repeated as an unexpected mixture is disclosed comprising commercially available compounds (see www.sigmaaldrich.com) for dietary, caring compounds, for cures which are then also commercially available. Further preparations (B+C) start from sterile media as well while those require technical skill and an equipment by preference for preparations of transdermal, pulmonal, cerebral, epimeningeal application forms. New biological extraction methods were disclosed with Applicant's prior preparations which are included here by citation (EP259939A1. Publ. Jun. 5, 2013).

The Procedure A Provides

(A1) apoproteins A (about 0.5 mg/ml) combined with liquidated albumin (1/1, vol/vol) as dispersion. Next, eco-certified acyl-phosphatidylcholines (500 nM), namely vitamin F and cysteine (30 mg/100 ml) are added at room temperature. The formation of lipidized particles is then promoted by means of cool temperatures and ultrasound pulsations (4° C.).
(A2) These vesicles combine themselves with lipid droplets of milk (≥20° C.) and their size is then reduced using ultrasound pulses (e.g. size from 3.5 μm to 70 nm, 4° C.) reaching a nanometer size for endogenous, intercellular passages and/or for passages across pores.
(A3) The eco-certified milk products are selected containing by preference 3.6 g proteins per 100 g (lactalbumin (60%) & lactglobulins) and 3.9 g/100 g lipids as an example of a favorable lipid-protein-quotient in a final product (FEQ: ≥1-1.4, pH 6.5) and those were
(A4) enriched with albumin-bound diacyl-phosphatidylcholines (about 500 nM), wherein especially cow milk is enriched with about 105 mg/100 g linoleic acids, linolenic acids because cow milk contains lower amounts of phospholipases compared to goat milk products containing lower levels of linoleic acids as well (vitamin F). In addition, minerals and vitamins are enriched (in mg: natrium 42, potassium 181, magnesium 11, calcium 127, iron 41, zinc 248, phosphate 109, iodine 4.1, vitamin E 0.1, vitamin C 2.0 mg, vitamin F (linoleic acid)) with the B-vitamins (in μg: thiamin 49, riboflavin 150, niacin 320, vitamin B12 70, folic acid 0.8) and the vesicles are then
(A5) coated with fatty lipid-phospholipid bilayers and are separated from supernatants at cool temperatures wherein especially saturated and/or unsaturated esterified C18-fatty acids are enriched by preference linoleic acids, omega-3-fatty acids, linolenic acids and wherein exterior encapsulations are enriched with plant butter, xanthine butter, cacao butter, Ginkgolide butter. The plant butter, cacao butter is enriched with dextrose (about 0.5 mM) especially for children.

The novelty of the processing (A) discloses that the milky preparation with eco-certified diacyl-phosphatidylcholines are protected for oral and dietary appliances against ingestion enzymes by means of coating, of capsules. Especially children, pregnant women, elderly persons need a neuro vegetative equilibration for improved cognitive capacities and for a smooth sleep without irritations. The dietary preparations (A) promote ingestions and can replace alcohol consumption as far as possible to antagonize hepatic problems. The milky proteophospholiposomes are further adapted for dermal, transdermal, inter-corneal passages, for a neuro vascular equilibration of the netlike microcirculation of the skin, the subdermis (Dermis, hypodermis) and/or for intercellular passages across epithelium cells of Plexus Pharyngeus, of Plexus Pulmonalis and/or of Chorioid Plexus. For oral, dietary, transdermal preparations with the milk are enriched with eco-certified acyl-phosphatidylcholines, with certified lecithin oils for oral, dietary, transdermal preparations and for the neuro vegetative equilibration of plexus systems for slight easing especially of children, against nocturiae, deficiencies of concentrations and enhanced agitations known as mild forms of ADHS syndromes.

A further embodiment of the inventive preparation is specified with the procedure B disclosing initial phosphate buffers whereby the successful cell protection is translated. Intact human cells are protected here against alcohol metabolites for the first time by means of preincubation with HDL-proteins prepared with ultracentrifugation and dialysis in the presence of delipidated albumin (final: 0.5 mg/ml HDL-proteins, 025% BSA). The unexpected protection of cells is reached especially with HDL-peptides in a freshly prepared HEPES-phosphate buffer (0.25% BSA, 11.9 mM NaHCo3, 137 mM Nacl, 2.68 mM KCL, 1 mM MgCl2, 041 mM NaH2PO4, 0.5 mM dextrose, 5 mM HEPES (containing sulfates). The HEPES-sulfate groups are replaced by cysteine (about 30 mg/100 g) and acyl-phosphatidylcholines are added (about 500 nM). The procedure can be performed with enriched own blood as well.

In Procedure B is Disclosed

(B1) that HDL-proteins are initially preincubated with intact washed thrombocytes in the presence of 0.25% albumin (3 min., 37° C., pH 7.4) in a HEPES-phosphate buffer. In the course of this 0.5 mg/ml HDL-proteins fusion with 0.25% delipidated albumin in the buffer (pH7.4, 2% NaHCO3, 1% amino acids, 1% folic acids, 1% RPMI vitamins comprising 11 vitamins of the B-group, with 0.1% vitamin E and 2% vitamin C). Then
(B2) certified organic lecithin oils are added (1/1, vol/vol) with enriched albumin (<10%). Cysteine is also added (about 30 mg/100 ml, 1% amino acids, 1% vitamins of the B-group, 1% folic acid, 0.1-2% vitamins of the vitamin groups A, C, E, D). The oily medium is then titrated considering the pH-values of the target organs. The plasmatic, lymphatic, meningeal compartments have neutral pH values (pH 7.2-7.4) while corneal dermal tissues have anionic pH values which are functionally important for skin barriers.
(B3) The above mentioned dispersion containing apoproteins A is mixed with the certified-organic lecithin oils and can be then placed back into the water in oil situation so as the HDL-like vesicles (a) transform themselves into micelles (b) which are then surrounded by further layers connecting the procedure B with the procedure A and/or C. The preparation steps can be done alone and/or together and can be sorted in an appropriate order whereby the steps are not limited thereto.

The Procedure C Begins

(C1) with the preparation of the unexpected inner HDL-vesicles wherein the apolipoproteins A fusion easily with albumin considering that albumin contains 17 cysteine groups (e.g. 3 minutes, 37° C.) and then
(C2) the novel peptide-composite is lipidized with certified lecithin oils containing 80% diacyl-phosphatidylcholines stabilized with cysteine groups (30 mg/100 ml), wherein the oily medium is enriched with 0.5 mg/ml apoproteins A, 0.25% delipidated albumin, 30 mg/100 ml cysteine, for example with di-palmitoyl-phosphatidylcholines (500 nM) and then the
(C3) formation of vesicles is promoted with ultrasound pulses (about 10 pulsations per minute, 4° C.) under sterile conditions for transdermal, parenteral, pulmonal, subcutaneous, epimeningeal application forms of vesicles (a) which can be further
(C4) titrated in a cationic manner e.g. with polyamines and/or with phosphatidylethanolamines (pH9.5) prior to further incubations with anionic peptides, attracting ionic micromaterials in a watery medium, whereby micelles (b) arrange themselves and are further titrated to even more anionic values using acetylcysteine and then
(C5) micelles are covered with lipids in a neutrally charged lipid medium.

The novelty is disclosed with the lamellar preparation of vesicles (a), micelles (b), liposomes (c) until the size of proteophospholiposomes is reached (d: 70-100 nm). Symmetrical and asymmetrical charges are used and/or are enforced whereby the modified ion exchange procedures of the priority document are included by citation.

The preparation using sterile media and synthetic, semisynthetic components is especially suitable for transdermal, subcutaneous, parenteral, invasive installations and/or for dosage forms promoting the meningeal drainage systems, comprising the plexus systems and the direct or indirect, the bidirectional, cerebral transport systems by preference the Chorioid plexus. The preparation steps can be performed alone, together, in the order above and/or in an adapted order as the preparations are not limited or limiting here. The modified ion exchange processes can be completed in an appropriate manner for separation, enrichment, purification of the proteophospholiposomes, whereby filtration methods and/or centrifugation methods can be used (≥500×g, 4° C.). The sterile preparation of inner HDL-vesicles is especially important for pulmonal, epimeningeal, parenteral and nasopharyngeal application forms reaching the Plexus Pulmonalis and/or the Chorioid Plexus. The neuro vascular, interstitial, cellular, cerebral application forms protect redox systems, ionic pulsations and the entire cell system (endothelial cells, epithelium cells, pericytes, glia cells, neurons, macrophages). Especially the nasopharyngeal application forms reach by means of direct or indirect pathways pulmonal endothelium which are then protected against fenestrations. The cerebral blood brain barriers are protected against influx-efflux problems and/or against reflux of before emigrated katabolic materials. The novel preparations containing transthyretin-prealbumins are enriched with cysteines and/or are e.g. titrated with acetylcysteine enhancing formation of a novel peptide composite containing apoproteins A and transthyretins.

### 6. PREPARATION OF DIAGNOSTIC TESTS AND TESTING PROCEDURES

A further embodiment of the novel proteophospholiposomes discloses the preparation of diagnostic devices and testing procedures (D). Especially the thiogroups are provided with electromagnetic markers. For example, methanonethiosulfonates (MTS) are commercially available. This marker is suitable for imaging procedures (NMR) by preference for diagnostic procedures concerning diastolic hypertension and/or for plexus diagnostic. The sulfate groups of cysteine covalently conjugate with nitroxide and clarify the diastolic hypertension at least in part, when those cases overlap here with too high values of anionic albumin and indicate then an irritation of the perivascular space, of the NO-vasodilation even among abstaining persons (Table 1). According to the invention, the proteophospholiposomes are thus prepared with a thiol-specific spin-label-marker prepared in the procedure C1.

In the preparation step C1 at least one cysteine group e.g. of albumin is provided with the thiol-specific spin-label-marker (MTS, biomol, USA) and then

(C2) combined with acyl-phosphatidylcholine, which are unlabeled or which are labeled with thiol-spin-label-markers as well and
(C3) are then prepared with HDL-like vesicles using the procedures A and B and are then lipidized by means of coating
(C4) comprising neutral lipid layers enriched with retinol for transcytosis by means of binding sites of transthyretin without irritations.

The labeled proteophospholiposomes are buffered, titrated to represent the transport by means of imaging procedures and/or to present the endogenous, xenobiotic cleavage of rest materials, which are activated by means of the methionine/cysteine-activated gluthation systems. The electrostatic capacity of the components is used for in vitro test devices and/or for testing procedures of the endothelium system as a whole, for neuro vascular interactions, for plexus systems and especially for the meningeal drainage system of the chorioid plexus.

A further diagnostic embodiment comprises the functional testing of albumin. Labeled albumin is then included in preparation step C1 by means of labeling a free C34-cysteine group with 4-maleimido as spin marker and is included to determine ligands of albumin and/or change of an albumin-configuration by means of vitro and in vivo testing e.g. with electron-paramagnetic resonance spectroscopy (EPR) for diagnostic tests especially of albuminuria and/or of liquor albumin, of alcohol-related hepatorenal syndromes. The age-related rise of vasopermeability is a higher risk class for a dysfunctional blood bran barriers which is verified below with cerebrovascular problems.

In addition, the electromagnetic tests and testing procedures of the priority document are included here by citation which are specified with the relevant preparation steps and which are completed with FiDA®-algorithms (Tables 1+2). The pro-diabetic risk profiles are determined here for the first time in an objective manner. Continued diagnostic tests are nevertheless required for a qualitative and quantitative ligand determination of transport proteins and especially of albumin. In vitro tests are disclosed for diagnostics of secretions, or urine samples, saliva, lacrimal fluid and/or of cerebrospinal fluids with the priority steps (P). An initial matrix-like electromagnetic surface is combined comprising a nonspecific fusion partner (PI) with a specific fusion partner (PII) for qualitative and quantitative measurements of proteophospholipids (PIII). Next, one or more pH-dependent fusion partners (PI) and/or the specific fusion partners (PII) are used in a labeled or unlabeled form whereby the labeled specific fusion partners (PII, PIII) are selected from the group comprising the soluble receptors against ether lipids/ether phospholipids and/or of the group comprising labeled antibodies against albumin or from the group comprising reactive components interacting with unfavorable ligands (e.g. with peroxides, alkyl-acyl-glycero-phosphocholines (Alkyl-GPC, PAF, LA-PAF, lysopaf)). The fusion partners PI, PII, PIII are labeled alone and/or together in a further step for example with spin-label-markers, with fluorescent markers, with colored, electric, electromagnetic markers, wherein labeled enzymes can be also used and are not limited thereto. The measurements are then performed comprising procedures, devices, subclasses, testing procedures, matrix-like surfaces, with charged surfaces, materials. For example, cationic surfaces are titrated with phosphatidylethanolamines and/or with polyamines (pH9.5) and bind then anionic materials. Neutrally to cationic charged materials reflect the zwitterionic acyl-phosphatidylcholines and bind native peptides and/or the novel anionic composite containing apoproteins A and the albumin-prealbumin group.

The test devices use electromagnetic procedures, spin-labeled markings for imaging processes, for laboratory methods including radioimmune assay, enzyme assays e.g. with phospholipases, phosphodiesterases and/or radiolabeled, colored, photometrical testing procedures suitable for tests, enrichment and/or for purification procedures of the novel proteophospholiposomes. HDL-like vesicles (a), micelles (b), liposomes (c) can be enriched, purified and/or included alone and/or together. The in vitro and/or in vivo testing of proteophospholiposomes is performed with appropriate detectors, whereby all testing probes, detectors can be used which are timely known by skilled persons comprising e.g. diagnostic, imaging procedures, spectrophotometric testing procedures, laser-scanning, for fluorescence microscopy, for microscopy by sight. The technical progress is provided with the presentation of the entire endothelium system, of luminal, perivascular matrix materials, of interstitial pulsations.

In accordance with the invention the proteophospholiposomes are applied as compound for health promotion and/or diagnostics and are adapted to physiological conditions to avoid irritations of the entire endothelium system. An equilibrated fat-protein-ratio (FEQ about 1.3) avoids then endogenous acidosis/ketosis. The layers of the proteophospholiposomes are titrated, buffered considering the endogenous pH values (plasma, lymph: pH 7.4; CNS: pH7.2; cytosol: about pH 7.0, stratum corneum pH 6.5-7.0). The fenestrated renal endothelium cells are used here as a representative model for fenestrated endothelium barriers around glands, in some parts of plexus capillaries including the Chorioid Plexus. Therapeutics and/or diagnostics preparations remain here in the nanometer area corresponding to the endogenous intercellular spaces and/or to pores of confluent endothelium barriers (about 20-100 nm).

The phospholipid membranes containing labeled or non-labeled thiolgroups can be used in vitro and/or in vivo for diagnostic methods of the luminal matrix materials, of interstitial spaces because the proteophospholiposomes increase their water content, stabilize endogenous pH values, improve ionic pulsations of the interstitial systems and protect fenestrated capillaries, by preference the gland capillaries and the endothelium layers of the liver sinusoids. The proteophospholiposomes allow expectation of non-physiological fenestrations during enhanced angiogenesis (diabetes, neoplastic disorders), during inflammatory pulmonal disorders. An increased proportion of fenestrated capillaries of the chorioid plexus is expected especially during hepatorenal syndromes. Especially the Chorioid Plexus determines the meningeal drainage systems and forms own transthyretin-prealbumin, counteracting the reabsorption of useless factors. The transthyretin transporters are combined here for the first time with apoproteins A and are lipidized as interior HDL-vesicles for diagnostic and/or cerebral appliances to equilibrate the Chorioid Plexus. The plexus systems modulate the neuro vascular, cholinergic systems comprising networks of capillaries, venules, lymph vessels and neuronal fibers especially the netlike plexus systems comprise perivascular, perineuronal, interstitial spaces and functions. The interstitial systems comprise the luminal matrix including heparan sulfates, the endothelium layers, the subendothelial basal membranes, the perivascular cells and spaces containing their extracellular glycoproteins. By preference the plexus systems are reached here by the proteophospholiposomes. The perivascular, the peri-intraneuronal spaces, the cells, the jelly-like fluids of the interstitial spaces are modulated by means of diffusion of ionic micromaterials, activate the ionic pulsations by means of pH values as well. The cleavage of useless materials is improved.

In accordance with the invention diagnostic and therapeutic proteophospholiposomes overlap adapting those to physiological conditions. The interior layers are provided with labeled or non-labeled thiogroups attracting ionic materials and cofactors. The core and/or the intermediate layers containing labeled and/or non-labeled albumin use a broad buffering capacity and bind symmetrically and/or asymmetrically charged ligands. The HDL-like vesicles are coated with neutrally charged lipid-phospholipid layers and the layers are buffered, titrated and/or labeled alone and/or together. As soon as the diagnostics indicate e.g. a non-physiological fenestration of an endothelium, declined matrix and/or overloaded albumin-transporters, the substitution is implicated by preference with the novel proteophospholiposomes completing nutritive compounds and cleaving of useless metabolites. Pharmaceutical interventions are prepared and/or completed in accordance with the medical guidelines. The technical progress of the new proteophospholiposomes provides smooth passages of diagnostics indicating influx-efflux problems without irritation of the endothelium system.

### 7. DIETARY APPLICATION FORMS OF THE NOVEL PROTEOPHOSPHOLIPOSOMES

An imbalance was found of HDL-dependent influx capacity of cholesterol esters versus efflux problems of VLDL in view of extensive population based examinations (Tables 1+2). In turn, a positive interaction was realized of circulating albumin with HDL-related influx capacities in view of healthy morning urines (Table 1). The albuminuria with diastolic hypertension (Table 2, phases I+II) is evaluated as prodiabetic, hepatorenal risk profile especially among alcohol consumers (Table 2, phases II+III). Albuminuria with normal blood pressure of elderly abstainers is interpreted as impaired matrix capacity (Table 2, col. 6). In accordance with the invention, the proteophospholiposomes are prepared here for oral, for dietary application forms, for protection and for equilibration of HDL-proteins and of the albumin-prealbumin group. A novel peptide composite is formed with albumin adducts and apoproteins A surrounded with acyl-phosphatidylcholines (e.g. 500 nM) trying to reach neutral pH-values considering fat-protein quotients (FEQ). The novel peptide composite is surrounded with several layers and the multilamellar proteophospholiposomes further remain in the nanometer size for diagnostic and therapeutic appliances. The novel peptide composite is lipidized and is used as HDL-like vesicles, which are surrounded with lecithins esterified with long chain fatty acids as well, for example with 105 mg linoleic acids per 100 g milk products or per 100 g plant butter. Milk products are used as watery medium in the above mentioned procedure A. Starting from the daily need of essential methionine which is 13 mg/kg (that means 650 mg/50 kg) and from the cysteine proportion of milk which is 30 mg cysteine per 100 g milk, an uptake only of milk products could hardly substitute the daily need of cysteine. On the other hand, the milk products of the above mentioned prescriptions provide sufficient amounts of calcium, natrium, potassium, magnesium, zinc, phosphates and the vitamins A, B, C, D, E, F. Cysteine is eventually substituted in milk products to better bind microelements and cofactors and to form acetyl Coenzyme A in the presence of B-vitamins. The proteophospholiposomes with choline groups effect then cholinergic systems and promote the formation of acetylcholines as tissue hormone. A neuro vegetative balance is reached by means of said plexus systems, by means of the plexus abdominals and this is important for neuro vegetative balance especially for children, pregnant women, for elderly persons with increased vasopermeability. Deficient conditions of children are often expressed as mental impairments, agitations, behavioral problems, psycho vegetative impairments, sleeplessness, nocturnal enuresis which can be improved at least in part by means of the cholinergic effective proteophospholiposomes of the invention. Enriched milk products are thereby carriers covering the daily need already with 500 ml per day without side effects. Overdoses are avoided then. Milk products can be recommended for persons with hepatic problems to replace the daily alcohol consumption if possible. The dietary proteophospholiposomes are coated with a neutrally charged exterior lipid layer, with xanthine butter, cacao butter and/or with ginkgolide butter and are sweetened with dextrose especially for children to antagonize restlessness.

### 8. DERMATIC, TRANSDERMATIC, COSMETIC COMPOUNDS, APPLICATIONS, PREPARATIONS OF/WITH PROTEOPHOSPHOLIPOSOMES

A preferred embodiment of the invention comprises the dermal, transdermal proteophospholiposomes as compound for unexpected appliances and for protection of the epidermis, dermis and subdermis, the neuro vascular networks and the lympho-vascular structures enclosing the subcutaneous fatty tissue as well. The novel proteophospholiposomes contain the novel peptide composite for the first time, adapted for dermal, transdermal appliances. The novel polypeptide composite arranges itself by means of cysteine groups and contains apoproteins A with at least one polypeptide from the albumin-prealbumin group. The anionic polypeptide composite is lipidized, coated, protected with at least one layer containing acyl-phosphatidylcholines. Moreover, these inner HDL-like vesicles are coated by preference with neutrally charged fatty components. These membranes contain triacylglycerols with saturated fatty acids, with esterified dipalmitoyl-oleic-glycerol as well (≥pH 7.0). One or more interior phospholipid layers contain by preference esterified long chain fatty acids from the group comprising oleic acids (C18-Acyl-GPC). These certified acyl-phosphatidylcholines (lecithins) are esterified by preference with unsaturated oleic acids such as e.g. linoleic acids, linolenic acids, with omeg-3-fatty acids extracted by preference from membranes using biological extraction methods. Starting materials are eco-certified plant butter, e.g. seeding oils, kernel oils, nut oils having rich proportions of omega-3-fatty acids.

The dermal, transdermal, cosmetic proteophospholiposomes arrange themselves again as layers especially due to the electrostatic capacities of anionic peptides, of cysteine groups and of zwitterionic and/or cationic phospholipids. Core and layers are enriched with cysteine groups, with cytidine phosphates which bind ionic micromaterials and which contain antioxidants such as vitamins retinol, tocopheroles, cholecalciferoles (vitamins A, B, C, D, E). The cytidine phosphates mediate growing, form coenzyme A and bind cofactors. These proteophospholiposomes pass intercorneal lipids, promote ceratine and hair growths. The xenobiotic cleavage of oxidized, of degraded, of alkylated lipids is stimulated by means of xenobiotic, thioclastic catabolism and promote cleavage e.g. of lipofuszines. The protein content of the proteophospholiposomes stabilizes anionic corneal pH values, whereby the corneal barrier can be strengthened (see filaggrin-recombinants from www.sigmaaldrich.com). By preference peptide-rich intermediate layers are prepared and/or subunits of vesicles (a), of micelles (b). Liposomes are combined microsomes together or combine separated microsomes in an outwardly united form of the proteophospholiposomes and/or in capsules. Several microliposomes can be further combined with exterior lipid layers for intercorneal passages in a united outer form. Overall, apoproteins A are combined with albumin as inner HDL-like vesicles for the first time for dermal, transdermal, cosmetic appliances and/or for direct installations. A relevant embodiment of the invention is the presence of cysteine and/or the cytidine phosphates. The cysteine groups are contained in the peptides and/or are enriched for sulfide bridges and/or for formation of Acetyl-Coenzyme A as growth factor e.g. in haircare products. The cytidine phosphates of the proteophospholiposomes connect the layers by means of electron clouds, sulfide bridges, ester condensations and further enrich the phospholipid layers with ionic micromaterials. The cytidine phosphates activate the thioclastic, xenobiotic purification systems of the skin and counteract aging spots, lipofuscins. The skin is purified, rejuvenated and is supplied with moisturizing factors and ionic micromaterials. The proteophospholiposomes are a reservoir and contain also albuminoid transporters in intermediate layers as delivery systems. The inner peptide composite remains coated with certified acyl-phosphatidylcholines. The formation of coenzymes A, of choline phosphates promotes acetylcholines and/or vasodilatory, protective hormones. The bio-reactive cytidine phosphates bind micromaterials such as e.g. silicates. The novel composite containing apoproteins A and the albumin groups for dermal appliances is then protected again with several layers and is further enriched with moisturizing factors which can then be enriched as subclasses, as vesicles (a), micelles (b) for retracted effects as well. Subunits are separated in a reversible manner and reach dermis, hypodermis with the glands, cells, reticular micro-vessels also of the subcutaneous fatty tissue. The moisturizing factors correspond with the natural factors of the young skin, comprising about a third of the natural volume of dermal cells especially of the corneocytes and keratinocytes. These moisturizing factors (NMF) contain about 5% sodium, about 6% chloride, about 4% potassium, about 1.5% calcium, about 0.5% phosphate, about 1.5% magnesium, about 1.5% creatine and 40% peptides, amino acids, glycoproteins adding here about 7% xanthine derivatives (urates etc.). Exterior layers of the proteophospholiposomes include materials with neutral pH values and/or lipids in the presence or absence of subunits. The novel proteophospholiposomes deliver the required moisturizing factors to the skin cells for regeneration e.g. of keratinocytes, melanocytes. In addition, the high proportion of proteins stabilizes the pH-values of the corneum (pH6.5<7) enforcing desmosomes, the skin barriers. The hair growths is promoted upon stabilization of keratin, of keratin-microfilaments. The novel proteophospholiposomes pass the intercorneal lipid layers and reach interstitial spaces (stratum corneum, lucidum, granulosum, spinosum, stratum basale). Nutritive components are delivered in this way including dermis and subdermis, whereby degraded products are then cleaved by means of the thioclastic, xenobiotic systems of cytidine phosphates. The cosmetic, topical, purifying care comprises the dermal papillae with their networks of capillaries, venules, arterioles, lymph capillaries and with the perivascular interstitial systems and the ionic pulsations of the jelly-like fluids. In addition, the fenestrated endothelium of the eccrine glands is protected. The hair follicles are stabilized equilibrating the secreting glands also in a hormonal manner by means of the cholinergic proteophospholiposomes. Moreover, degraded secretions are cleaved such as sebum and perspirations without unfavorable changes of the pH values, e.g. known from detergents. The novel proteophospholiposomes reduce swelling, enhance cleavage of aggregates by means of the (peri-)vascular, plexus-like networks of the dermis and hypodermis. The endogenous purification of the skin is activated without detergents. Hair follicles are stimulated by means of natural growth factors, with cytidine phosphates. The skin care comprises the skin of the face, of the body, hairs and nails by means of natural moistening factors comprising mostly amino acids and peptides composed here with one or more layers which are integrated in the novel proteophospholiposomes. Fatty thio-phosphatidylcholines stabilize the exterior lipid membranes as well of the novel proteophospholiposomes. Cytidine phosphates form acetylCoenzyme A as growth factor for the dermal, transdermal care, for rejuvenating purification of skin barriers, of skin cells (corneocytes, keratinocytes, melanocytes). The interstitial systems are cared including eccrine glands of sebum and perspiration. The perfusion is promoted of the interconnected vessels of the skin. The purification of pores, of epidermal sebum glands, of the dermatic hair follicles enforcing keratin, keratin-fibrils, hairs, nails by means of cysteine groups enabling an endogenous self-purification of the skin without detergents. The cytidine phosphates, thiamin phosphates promote choline phosphates as cholinergic substrates of acetylcholines, as hormonal compound of the skin inter alia against hormonal pigmentation problems and/or against vasoconstrictions. The novel cell protection with apoproteins A antagonizes age-mediated skin problems and cytidine phosphates, choline phosphates of the phospholipid layers improve the membranes of skin cells by means of better phospholipid profiles. The cytidine phosphates are natural growth factors and counteract wrinkling, the hormonal problems, forehead boldness and/or age-related skin problems (aging spots, lipofuscins, amyloids, age-related verrucae) and promote the regeneration of the entire skin.

For the first time, HDL-peptides are disclosed here as a composite containing the albumin-prealbumin group for dermal, transdermal, cosmetic proteophospholiposomes coated with certified acyl-phosphatidylcholines and those are by preference esterified with oleic acids, linoleic acids, linolenic acids (≥4% C-18-Acyl groups). The effect of the proteophospholipids can be enhanced by means e.g. of subcutaneous application forms and/or with non-invasive stimulations (e.g. vacuum treatment, skin masks etc). Plexus-like vascular networks can be reached even better in this way comprising capillaries, venules, lymph and perivascular interstitial structures especially in the hypodermis which delivers the subcutaneous lipid tissue as well. Especially the luminal matrix barriers, the capillaries including the subendothelial cells are protected by means of the HDL-like vesicles, which cleave also wasted cholesterol esters by means of the ATP-dependent transmembrane efflux systems.

The direct protection of cells is shown here with own unpublished data because HDL-proteins (0.5 mg/ml) protect here for the first time intact washed platelets in the presence of delipidated albumin (0.25%) in a HEPES-buffer which is titrated as representative model (pH7.4). The protection of phospholipid membranes of human cells is directly translated here to the protection of keratinocytes, of melanocytes and of monocytic cells such as e.g. endothelium cells, macrophages, adipocytes. The cosmetic, topical, purifying protection comprises the skin cells including endothelium cells, subendothelial cells, corneocytes and macrophages. The papillae with their microvascular networks, the lymph capillaries, the interstitial fluids contain fenestrated endothelium at least in the areas around skin glands and in luminal parts of vessels which are protected with the proteophospholiposomes as well. In addition, the hair follicles are stabilized with cysteine and are supplemented with nutritive components whereby the secreting glands are equilibrated in a hormonal manner as well. Sebum and perspirations of the eccrine glands are cleaved. The proteophospholiposomes protect membranes of cells, intracellular membranes, organelles and/or the DNA. The proteophospholiposomes promote the anionic, corneal pH-values and decrease undesired swellings. The unidirectional lymph flow promotes regeneration of the (peri)vascular, plexus-like networks of the dermis and hypodermis. At least one intermediate layer contains albumin with cysteines and provides a broad buffering capacity protecting against swelling of the skin. Cofactors are enriched e.g. xanthines, filaggrins and silicates which promote the hair growths. A special technical progress is disclosed with cell protection combined with an endogen purification of the skin in the absence of detergents, whereby the skin comprises face skin, body skin, hairs and nails.

The preparation of the novel, buffered, titrated proteophospholiposomes starts here with the stationary media (see e.g. procedure B). The oily media contain by preference certified organic acyl-phosphatidylcholines. The protein-rich media contain the electrolytes of the experimental buffer system and by preferences albumin with bound apoproteins A. Cysteines activate the acetyl groups, ionic micromaterials and antioxidants. The media are buffered, titrated and can be used as carriers upon enrichments with the proteophospholiposomes. The proteophospholiposomes enrich themselves layer by layer during the incubation and upon optional changes of oily and watery media. The desired components are integrated and enriched by the proteophospholiposomes using the extraction methods as specified hereinafter. A facilitated option applies commercially available, synthetic, semisynthetic products, recombinants (see www.sigmaladrich.com).

The preferred extraction methods composed here esterified long chain acyl-phosphatidylcholines, whereby the biological extraction methods start with sedimented membranes and with eco-certified starting materials in accordance with Applicant's prior publications. Membranes from the group of plants, animals, sea weed, plankton are prepared at cool temperatures with physical methods and are prepared with Applicant's biological extraction methods (see Ruth-Maria Korth, EPA-Publ. EP2 599 393A1). The sedimented membranes are dissolved with synthetic dipalmitoyl-phosphatidylcholines (C-16) and are extracted at cool temperatures over longer time periods (>24 hours). Titrated palmitoyl-phosphatidylcholines are dissolved as dispersions using ultrasound pulses (about pH 6.5). The procedure is repeated after long time incubations so as the palmitoyl-phosphatidylcholines are enriched with linoleic-phosphatidylcholines (>4° C.). The formation of vesicles is enhanced by means of ultrasound pulsations at cool temperatures and those are by preference enriched and purified with the modified ion exchange processing of the priority document. The fatty acyl-phosphatidylcholines are esterified with increasingly lipophilic acyl groups and are used here as oily stationary media to enrich proteophospholiposomes layer by layer. Vesicles form themselves having phospholipid layers, phospholipid bilayers. The zwitterionic phosphatidylcholines can fusion with anionic peptides by means of electrostatic properties. The anionic peptide composite is lipidized, surrounded, protected. One or more exterior lipid layers of the proteophospholiposomes can be enriched with xanthine butter, with cacao butter and/or with ginkgolide butter. The passages across corneal skin layers are possible with compounds not containing here fatty alcohols, without flavonoids, without essential oils to avoid pigmentation problems. The inner vesicles contain the composite comprising apoproteins A and albumin and are coated with layers containing certified acyl-phosphatidylcholines which are further protected with thiogroups against transformation, so as the entire skin is protected and supported.

### 9. THERAPEUTICAL COMPOUNDS FOR INVASIVE AND NON-INVASIVE INSTALLATIONS

The currently evaluated risk profiles show that chronic alcohol consumption and tobacco smoking overlap and those often overlap with an impaired endothelial protection system as well. The impaired protection system is verified here with the novel FiDA®-algorithms in an objective manner (Tables 1+2). Persons with chronic alcohol consumption are in a higher risk group during virus disorders especially during pulmonal disorders. The impaired defense capacity comprises the entire endothelium system, wherein the pulmonal capillaries are at risk due to the near epithelium of alveoli because anti-atelektesis factors protect said neighborhood of healthy persons. The major proportions of these surfactant factors comprise dipalmitoyl-phosphatidylcholines (80% Surfactant lecithins) enhancing self-purification of the lungs and counteract alveolar collaps, reducing the surface tension and an overactivation of alveolar macrophages.

In accordance with the invention, the proteophospholiposomes are prepared for pulmonal appliances to supplement consumed surfactant factors implicating supplementation of proteophospholiposomes to protect pulmonal cells in general. The proteophospholiposomes are prepared for pulmonal appliances with apoproteins A and at least one peptide of the albumin-prealbumin group, which are fusioned using acetylcysteines and contain especially synthetic dipalmitoyl phosphatidylcholines (80%). Vesicles are lipidized and are coated with neutrally charged lipids e.g. with cholesteryl stearates (<10%). The interior peptide-composite contains apoproteins A with albumin and HDL-like vesicles which are enriched with acetylcysteines. The broad buffering capacity of albumin is used for thioclastic capacities of the cysteine groups. Especially mucolytic acetylcysteines are enriched. Ionic micromaterials are enriched containing by preference the calcium-group, the phosphate group and cholecalciferoles.

The novel peptide composite is coated with synthetic dipalmitoyl-phosphatidylcholines. These proteophospholiposomes for pulmonal application forms are suitable for a complementary therapy e.g. during treatment of severe pulmonal disorders, during treatment with antibiotics, with dexamethasones. Less suitable are the commercially available, sterile extracts from pulmonal extracts originating from cows or pigs for direct installations (e.g. 200 mg/kg, once in 12 hours). This treatment is replaced here using synthetic dipalmitoyl-phosphatidylcholines. The commercially available, purified extracts from animals should be replaced by the novel proteophospholiposomes. Especially children should obtain by preference synthetic dipalmitoyl-phosphatidylcholines. Appropriate pharmaceutics should be completed in accordance with the guidelines.

A further embodiment of the novel proteophospholiposomes are the preparations for ophthalmological appliances whereby synthetic dipalmitoyl phosphatidylcholines are preferred here and/or certified phosphatidylcholines, containing about 13% palmitoyl fatty acids and more than 4% stearic fatty acids. The inner HDL-like vesicles are again composed with cysteine groups. Apoproteins A are conjugated by preference with transthyretin for lacrimal application forms. The biologically active cytidine phosphates arrange themselves easily with dipalmitoyl-phosphatidylcholines, surrounding the HDL-like vesicles. The lacrimal application forms can reach the choroid plexus by intermediate of the sinus cavernosus and are protected for the passages in an appropriate manner.

The apoproteins A can be applied as commercially available recombinant peptides as composition with certified human albumin (50 g/l human serum albumin per infusion). Oily proteophospholiposomes are specifically prepared for ocular appliances need almost no covering. The preferred ocular application forms are tear fluids, ocular oils, ocular droplets. The ocular proteophospholiposomes can contain the hydrophobic glycoproteins of the surfactant group in the core (SP-B and SPC). The dipalmitoyl-phosphatidylcholines (80%) are surfactant factors also for ocular application forms and are prepared with biological extraction methods which are specified together with the modified ion exchange processing of the priority document. One or more layers contain then the certified dipalmitoyl-phosphatidylcholines. Surfactant proteins contain half plasma proteins and are administered here with the inner HDL-like vesicles. Complex glycoproteins can be completed when pure peptides are available (surfactant peptides A, B, C, D).

The bronchial installations of sterilized animal extracts are risky especially for children. The surfactant factors decrease during acquired pulmonal stress syndromes among adults as well e.g. during virus pneumonia. The proteophospholiposomes as a complementary cell protection, for protection of the entire endothelium system implicating protection of capillaries in the presence of infected bronchi and alveoli in their neighborhood. The ionic micromaterials diffuse into the interstitial spaces of near bronchi. The alveoli are then relieved, the distance between capillaries and epithelium is increased and a dangerous fenestration of pulmonal capillaries is antagonized as far as possible. The alveolar antiatelektase factors protect cells, endothelium cells, epithelium cells and alveolar macrophages. The surfactant factors are repaired and their consumption and/or fenestration of pulmonal capillaries are counteracted as major risk factor during collapsing alveoli. A collapse of alveoli can be further antagonized by means of positive ventilation pressure. The novel pulmonal proteophospholiposomes further contain bio-reactive cytidine phosphates supporting the formation of choline phosphates and acetyl-coenzyme A and promote cholinergic agonists especially by means of the cholinergic binding sites of alveolar macrophages to counteract overexpression of immune reactions. The cysteine groups activate thioclastic, xenobiotic cleavage systems and relieve alveoli in the neighborhood e.g. upon cleaving of peroxides. The cysteine groups require antioxidants which are substituted especially with the albumin adduct containing then vitamins A, B, C, D, E. The interior vesicles can be surrounded with an exterior lipid bilayer containing semisynthetic or synthetic phosphatidylcholines. Overall, the dipalmitoyl-phosphatidylcholines are enriched and stabilized with cysteine groups, with cytidine phosphates of the methionine-cysteine group enhancing the formation of acetylcholine with intermediate of coenzyme A and choline phosphates. The cholinergic subclasses of monocytic receptors are balanced including also the pulmonal plexus systems. The thio-groups are donators and acceptors of ionic micromaterials which promote the luminal matrix and which diffuse so as perivascular spaces attract water, increase the ionic pulsations and the distance between endothelium and epithelium to prevent fenestration of pulmonal capillaries in time. The proteophospholiposomes containing 1,2-palmitoyl-phosphatidylcholines and cholesteryl stearates can be easily prepared with commercially available synthetic materials e.g. with the preparation as specified above with procedure (B) (www.sigma-aldrich).

In accordance with the invention, synthetic substances are prepared for direct installations, for invasive procedures with needles, infusions, plasmapheresis, bronchial pheresis, dialyses. As direct installations e.g. broncho-alveolar lavages, intrabronchial installations are used accompanied by near ventilation options. The intratracheal, intrabronchial installations are applied during severe ventilation problems, during invasive procedures also throughout positive ventilation pressure of children. The invasive, subcutaneous, parenteral installations can complete then immune-reactive compounds, steroids, antibiotics and/or therapeutic procedures with antibodies directed against adhesion molecules (e.g. CD200, CD47)).

The noninvasive procedures are developed here by preference for therapeutics of elderly persons, pregnant women and children. The transdermal, transmucosal, pulmonal, nasal lymphatic, retropharyngeal applications can use all known pharmaceutical carriers as well. The carriers are then also titrated and buffered e.g. with natrium-hydrogen-carbonates. The cytidine phosphates activate lymphatic, interstitial, xenobiotic systems and cleave harmful ligands in the presence of equilibrated redox-systems. The cleavage e.g. of peroxides protects then cells, organs, barriers, tissues whereby the endothelium system and the alveoli are treated here as neighbor tissues. The noninvasive dosage forms with proteophospholiposomes reach the appropriate plexus systems, by preference the Plexus Pulmonalis comprising the microvascular networks which can be reached by means of flow vectors in the Plexus Pharyngeus upon oral, pharyngeal, buccal, nasal, therapeutical dosage forms as well. The transdermal installations, the subcutaneous, parental therapeutics can be completed and/or replaced as well. The novel proteophospholiposomes can be prepared or dosed as tablets, as effervescent agents, dispersions, inhalations, sprays, as oily compounds. The dermal, dietary, pulmonal, pharmaceutical appliances can be combined with all known pharmaceutical carrier systems. All known application forms can be used which improve a bioavailability and/or prolong the stability of the new, unexpected proteophospholiposomes especially for the pulmonal supplementation of surfactant-phospholipids, of dipalmitoyl-phosphatidylcholines, for pulmonal appliances.

The preparation of novel, pulmonal and ocular compounds and appliances is not limited to the specified procedures as well. The innovation comprises the protection of cells by means of apoproteins and albuminuoids which are coated with synthetic dipalmitoyl-phosphatidylcholines.

### 10. PROTEOPHOSPHOLIPOSOMES FOR PROTECTION OF NEURONAL CELLS, FOR PROMOTION OF LYMPHO-MENINGEAL DRAINAGE SYSTEMS

A preferred embodiment discloses proteophospholiposomes that help protection of neuronal cells and for promotion of lympho-meningeal, epimeningeal drainage systems. The development was based on shown increase of vasopermeability among elderly persons and/or of alcohol consumers (Tables 1+2). A commination of albuminuria with diastolic hypertension indicate a negative interaction of endothelial and subendothelial cells and progression can be hardly limited to defects only of peripheric, hepatorenal cells especially of alcohol consumers. The trans-subendothelial problems during increased values of plasma albumin of abstainers can be mostly cured by means of equilibration of shifted pH-values, of dehydration and/or of malnutrition. On the other hand, critical FIDA® algorithms verify manifest cellular defects mostly during chronic alcohol consumption. The proteophospholiposomes are then prepared as alcohol-free, drinkable liquids and are composed for supplementation of malnutrition, of fluid deficit and for supplementation of electrolytes. Energetic compositions brighten up and facilitate periods of alcohol restriction and/or of nicotine restriction containing for example xanthines. The novel proteophospholiposomes are prepared and buffered layer by layer. The endogenous pH-values are considered of interstitial, of cerebral systems (about pH 7.2) whereby pH values of the added components are summarized and adapted in accordance with their isoelectric pH values. The proteophospholiposomes are initially prepared containing the inner HDL-like vesicles which are then adapted to the conditions of the blood brain barriers considering their closed areas, whereby luminal matrix covers the endothelium barriers which are closed with pH-insensitive gaps (desmosomes) and which are directly connected by means of cell processes (“endfeet”) with subendothelial cells comprising astrocytes, glia cells, pericytes, neurons and macrophages. The HDL-like vesicles contain a new peptide composite for cerebral applications whereby apoproteins A are conjugated with at least one factor from the transthyretin-prealbumin group and whereby the inner HDL-like vesicles are coated with one or more additional layers containing certified acyl-phosphatidylcholines. An intermediate layer is prepared for relief of the meningeal albuminoid transporters containing albumin adducts which connect the inner HDL-like vesicles with outer coverings by means of their anionic capacities as the exterior layers contain by preference neutrally charged lipids. Albumin is recognized as decisive meningeal acceptor of excreted cerebral materials for the first time as shown with yet unpublished statistical data which are specified below. The size of the proteophospholiposomes is reduced during endogenous passages so as the outer layers are effective in the luminary, capillary, lymphatic, extracerebral, meningeal compartments. The inner HDL-like vesicles are prepared to equilibrate the influx-efflux systems of the blood brain barriers including their luminal and subendothelial areas. At least some areas of the capillaries of the cerebral Chorioid plexus are fenestrated as shown in the representative model of FIG. 2.

The novel protein composite in the interior HDL-like vesicles is thus prepared by preference with apoproteins A and transthyretin-prealbumins whereby sulfide bonds are triggered by addition of cysteine groups. The composite containing apoproteins A and transthyretins is titrated and the redox homeostasis is balanced with vitamins e.g. with tocopheroles. The proteophospholiposomes further contain an anionic intermediate layer with albumin that is enriched with cysteines and layers arrange themselves with one or more outer lipid layers by means of electrostatic capacities. The proteophospholiposomes then contain apoproteins A and transthyretin-prealbumins as HDL-like inner vesicles which are coated with certified acyl-phosphatidylcholines and which are stabilized with thio-phosphatidylcholines and with ionic microelements to activate also choline groups which have cholinergic effects due to choline phosphates. The acetylcholine(re)synthesis is enhanced by means of acetylcoenzymes A as cysteine-derivative. The composite containing apoproteins A and tranthyretin-prealbumins is thus surrounded with one or more layers containing zwitterionic acyl-phosphatidylcholines and with an intermediate layer containing peptides, glycoproteins, cofactors such as reelin and vitamins such as retinol. In addition, e.g. neutrally charged lipid layers further protect the application forms especially those for epimeningeal plexus passages. The inner HDL-like vesicles can be directly administered without exterior lipid coats e.g. by means of meningeal injections, catheter, pheresis, with nocturnal perfusions e.g. during meningeal pheresis considering that the nocturnal vector of the meningeal drainage systems can turn around. For noninvasive procedures the outer lipid layers are titrated and buffered as well especially with B-vitamins so as cytidine phosphates can form acetylcholines in the presence of choline phosphates for neuro vegetative equilibration, for promotion of cholinergic transmitters modulating GABA-receptors. The noninvasive applications of the cholinergic proteophospholiposomes antagonize agitation problems, insomnia and/or concentration impairments especially of children, pregnant women and/or memory impairments of elderly persons without side effects and without risk for addiction. In addition, the proteophospholiposomes facilitate the restriction from alcohol of persons having alcohol problems.

In accordance with the invention, the noninvasive dosage forms are preferably used for cerebral applications which can further promote the cerebral-meningeal drainage systems. The microcirculatory networks are used starting e.g. from Plexus Pharyngeus as the proteophospholiposomes are catabolized layer by layer starting from exterior layers and then inwards. The endogenous passages can be then shown e.g. with imaging procedures. The proteophospholiposomes are labeled layer by layer for this purpose e.g. by means of the cysteine groups in albumin and/or with thiolgroups using spin label markers. The novel composites containing apoproteins A with transthyretin-prealbumins reduce the re-absorption of excreted cerebral metabolites which are then cleaved in a thioclastic manner by means of albumin adducts containing cysteines. About 1% of peripheric albumin is measured here in the cerebrospinal fluid (CSF). The Chorioid Plexus itself forms transthyretin for inhibition of re-absorption of excreted metabolites which is distinguished below from renal tubular reabsorption which can lead to tubular fibrosis due to overloading. An intermediate albumin-layer is thus prepared which can be administered with nasal oils as well. Albumin can be selected from the group comprising serum albumin, the endurable whey proteins, the lactalbumin with lact-globulin as starting material. Sterile synthetic, semisynthetic peptides and certified acyl-phosphatidylcholines (PC) should be preferred for direct and/or invasive installations. The inner vesicles be protected as far as possible against transformations and also against degradation during the endogenous passages. Semisynthetic and synthetic peptides, amino acids, acyl-phosphatidylcholines and cysteines are commercially available. The apoproteins A (apoA) are available as recombinant products. For the first time inner HDL-like vesicles are stabilized here and are then further protected by exterior layers suitable for activation of cleavage systems. The multilamellar proteoliposomes deliver ionic micromaterials reaching then plexus systems e.g. by means of nasopharyngeal applications and the diffusion of micromaterials then improves the perfusion. Especially albumin of the intermediate layers is then donator of ionic micromaterials and acceptor of emigrated metabolites and/or of useless materials. Acetylcoenzymes A are formed and reach the cholinergic structures and filaments in/of Sinus Cavernosus. The neurovascular networks in plexus systems of the head are connected to each other so as proteophospholiposomes can reach Sinus Cavernosus by means of nasopharyngeal, lacrimal, epimeningeal applications whereby the cholinergic structures and filaments promote the neuro vegetative balance of the cerebral blood-flow. In addition, the thioclastic, xenobiotic cleavage system is promoted directed against useless materials in the cerebral and extracerebral, in the neurovascular networks of the plexus systems in the head. Moreover, the interior HDL-like vesicles pass the blood brain barrier using specific transport systems of otherwise closed endothelium barriers. The bidirectional transport systems of the cerebral Chorioid Plexus comprise fenestrated areas as minor part of the cerebral capillaries while endothelium layers are mostly confluent expressing pores which allow free diffusion at least of micromaterials (see FIG. 2). The interior HDL-like vesicles further reach the subendothelial efflux systems comprising functional VLDL-Apoprotein E receptors promoting then efflux systems in combination with the reelin system. For transcytosis the HDL-like vesicles are enriched here with retinol interacting then with transmembrane transport systems of transthyretin.

The doses of the proteophospholiposomes considers that a physiological transcytosis is limited to about 1% of the circulating HDL. The amount of HDL (0.5 mg/ml) is appropriate in the yet unpublished experiment protecting here intact human cells (FIG. 1). In accordance with the invention, clinical observations are based on pathophysiologal pathways of fatty alcohols which were transposed to reach a therapeutic balance of cholinergic neuro-vegetative systems and for cleavage of unhealthy alcohol metabolites. The protection comprise endothelium cells, perivascular cells, cells of organs and tissues and also astrocytes, pericytes and neurons as well. The luminal matrix materials of the blood brain barriers are protected as well. A small portion of the plexus capillaries comprises fenestrated endothelium barriers. The capillaries of glands and/or of the diabetic eye background including angiogenesis are also fenestrated so as the size of the particles is adapted to the size of the intercellular spaces which is in the nanometer size (see FIG. 2). The evaluation of risk profiles is specified with appropriate terms with the Tables. The luminal matrix materials are protected because cytidine phosphates can directly interact with heparan sulfates protecting matrix membranes which modulate the cerebral influx-efflux-systems as well. The pH-values of the proteophospholiposomes are adapted and the lipid-protein-ratio (FEQ) is balanced to avoid a change of the cerebral pH-values (pH7.2). The lipids and phospholipids are adapted to the amount of peptides to avoid an acidosis (e.g. 0.9% lipids to 0.7% protein per 100 g is a FEQ of 1.4). The certified acyl-phosphatidylcholines of the proteophospholiposomes are stabilized with thio-ether-phosphatidylcholines protecting against oxidation and degradation due to proteinases, lipases, phospholipases and bind micromaterials, antioxidants and cofactors. The inner HDL-vesicles promote, complete the capacities of the HDL-related influx-efflux-systems and trigger also the luminal ATP-dependent transporters. In addition, the outer lipid layers are enriched with cholecalciferoles, xanthine butter and/or Ginkgolide butter for promotion of the luminal cAMP-regulatory response elements. The apoproteinsA enter the cerebral system by means of the lipoprotein receptor family and/or efflux is promoted by means of VLDL-apoprotein E-reelin systems which are connected with the cerebral efflux systems e.g. of cholesterol esters and the cerebral efflux systems are connected with ATP-transporters (see FIG. 2). Overall, the apoproteins A protect here for the first time the luminal and the subendothelium cells by means of diffusion of the ionic micromaterials and their composition is disclosed with the yet unpublished experiments (see legend of FIG. 1). Diffused ionic micromaterials spread and the proteophospholiposomes improve therewith interstitial cerebral systems, the fluidity of the perivascular, perineuronal spaces. The regeneration is supported of the endothelium system as a whole. The proteophospholiposomes balance said neuro vegetative systems and promote bidirectional transport system of the Chorioid Plexus. The outer layers promote the luminal efflux systems wherein cAMP-dependent influx systems are supplemented here with albumin as luminal acceptor of exudation materials especially in the extracerebral, meningeal drainage systems whereby albumin containing the cysteine groups enhance the thioclastic degradation. In accordance with the invention, the entire influx-efflux systems are promoted and especially the bidirectional transporters of the cerebral Chorioid Plexus.

The noninvasive application forms are preferred and not limited here. The topical, dermal, transdermal, lymphatic, oral, pharyngeal, nasopharyngeal, nasal, lacrimal, ophthalmic, buccal, sublingual, epi-meningeal application forms reach the appropriate plexus systems of the head. The preferred but not limited application forms, the carriers of the proteophospholiposomes are selected, for example nasolymphatic compounds from the group comprising the oils, the dispersions, the solutions, the cremes, the lipids, the sprays, the inhalation compounds for non-invasive, mediated installations. Dermal, transdermal, cosmetic carriers can also balance in a neuro vegetative manner especially by means of oily carriers. The preferred but not limited application forms are dispersions, oils, cremes, lipids for oral, buccal, lingual, sublingual dosage forms reaching the Plexus pharyngeus with entry of the Sinus cavernosus and then the Chorioid plexus by means of the interstitial pulsations. Intranasal installations and/or oral, nasal, lacrimal, dosage forms, washes of the paranasal sinuses, retropharyngeal washes can also reach the microvascular, neurovascular networks of the plexus systems and can effect cholinergic filaments and receptors to improve the neuro vegetative balance. The microvascular networks of the Sinus cavernosus reach the cerebral Chorioid Plexus mediated by perivascular spaces and ionic pulsations. Especially the cerebral Chorioid Plexus is the preferred target containing networks of capillaries, epithelium, nerve filaments. The interior HDL-like vesicles reach the cerebral bidirectional transporters and about 1% of apoproteins A is admitted by means of specific luminal transporters and about 1% of the plasmatic albumin enters the meningeal drainage systems and binds fatty alcohols. The below mentioned, yet unpublished statistics in the chapter 12 confirm the clinical estimates as specified in chapter 11. The preferred and not limited embodiment of the carriers, of the proteophospholiposomes are selected for example from compounds from the group comprising the oils, the dispersions, the solutions, the cremes, the lipids, the sprays, compounds for inhalations. The dermal, transdermal, cosmetic compounds comprising applications on upper lips selecting appropriate carriers here for the first time, for example as cream sticks, lip sticks and are not limited thereto. Dispersions, oils, cremes, lipids are used for oral, buccal, lingual, sublingual dosage forms of proteophospholiposomes to reach the Plexus pharyngeus, the Sinus Cavernosis so as the interior HDL-like vesicles reach the interstitial networks of the Chorioid Plexus. Intranasal installations and/or oral washes with proteophospholiposomes reach cholinergic systems, filaments, receptors and can contribute to neuro-vegetative balances and to psycho-vegetative stabilization. Especially the plexus systems connect the cholinergic elements with the bidirectional influx-efflux systems of the entire blood brain barriers, whereby the Chorioid Plexus connects endothelial and epithelial structural elements. The perivascular astrocytes together with the glia systems regulate the formation of apoproteins E, the fluidity of the interstitial gels and deliver nutrients to cholinergic neurons by means of cerebral gluconeogenese and of GABA-receptors which are promoted here by means of the proteophospholiposomes delivering nutritive compounds and cleave useless materials by means of uniformed HDL-type systems.

A special embodiment of the invention concerns the novel tests and testing procedures of the priority documents measuring ligands of overloaded transporters. Overloaded carriers are clinically realized here with the critical influx-efflux systems which are verified with the novel FiDA® algorithms in an objective manner (Tables 1+2). Relevant influx-efflux-problems and/or the improved tests of the priority document facilitate follow up examinations during supplementations of substrates administered with the inventive proteophospholiposomes.

Own unpublished data show here an about two hundredfold higher concentration of plasmatic albumin compared to CSF-albumin in cerebrospinal fluids (here 19±12 mg per dl CSF). A stable meningeal uptake of albumin was shown here as being 0.5% under healthy conditions and increases here to about 1% during inflammations. Only three of thirty persons in a specialized hospital showed here neurological and inflammatory symptoms together with increased albumin levels in cerebrospinal fluids (45±14 mg/dl>30 mg/dl CSF-albumin, 3 of 30). The increased levels of albumin in cerebrospinal fluids failed a direct correlation with mental symptoms while a relevant multifactorial relationship is shown (p=0.06). Albumin is a meningeal acceptor of excreted lysopaf which is chemically an alcohol metabolite and is tested with yet unpublished statistical values in chapter 12. Regarding urinary albumin (0.3-3 mg/dl) an emigration of 0.01% could be assessed compared to plasma albumin (4000 mg/dl). The emigration rate of plasmatic albumin into the liquor was assessed with 0.1% of peripheric albumin. This difference was estimated here as renal, tubular reabsorption of albumin having a magnitude which would overwhelm the bidirectional transport systems of the Chorioid Plexus. In accordance with the invention the possible meningeal re-absorption is reduced as far as possible by means of the novel proteophospholiposomes comprising at least one intermediate layer containing albumin adducts with cysteines for the thioclastic, lymphomeningeal cleavage. Albumin is placed into at least one of the layers which are catabolized during the endogenous passages following noninvasive application forms. The data in the chapters 11 and 12 show the requirement of an improved testing of albumin adducts, and the need of the relevant proteophospholiposomes which are prepared with novel electromagnetic tests and new testing procedures based on the priority document. An improved diagnostic is required of albumin in secretions, eye fluids, in the nasopharyngeal space and/or in the liquor. The albuminoid transporters cleave useless metabolites and catabolize fatty alcohols by means of activated thioclastic systems for the first time including cleavage of toxic ether phospholipids and/or amyloids, of useless proteins including harmful aggregates as well (β-amyloids, tau-proteins etc). Significantly higher values of lysopaf per mg albumin in the cerebrospinal fluids were correlated already before with psychotic disorders (see U.S. Pat. No. 5,605,927, Ruth-Maria Korth, 1997).

In accordance with the invention, apoproteins A are conjugated for the first time with transthyretin and are coated with neutrally charged layers containing certified acyl-phosphatidylcholines to protect for the first time the neurovascular system, the cerebral influx-efflux-systems. The novel proteophospholiposomes antagonize risk profiles during increased levels and nutritional failures or during alcohol problems. The risk profiles are relevant regarding the overlapping problems, the diastolic hypertension and/or the dyslipidemic, prodiabetic, alcoholic, hepatorenal problems (Tables 1+2). The proteophospholiposomes supplement consumed transporters related with the meningeal drainage systems as well. In addition, the proteophospholiposomes are completed with cofactors which are selected from the group comprising glycoproteins, the antagonists against fatty alcohols, against alkylated lecithins, against alkyl-GPC (PAF, Lysopaf). Aggregates are dissociated in a thioclastic manner including also dysfunctional amyloids, β-amyloids, hyperphosphorylated TAU-proteins, toxic metabolites. Cytidine phosphates and B-vitamins further promote the (re)synthesis of acetylcholines by means of acetylCoenzymes A and choline phosphates wherein the cholinergic (re-)synthesis of acetylcholines is connected with the calming cAMP-dependent GABA-systems for promotion of calmness and sleep without risk for addiction. The health is promoted balancing cerebral influx-efflux systems.

### 11. RISK PROFILES AND OBJECTIVE FIDA®-ALGORITHMS

An important embodiment of the invention is provided with the novel FIDA® algorithms showing an imbalance of HDL-related apoproteins A versus VLDL-related systems. It was found before that especially apoproteins B in VLDL and LDL lipidized human cells in the presence of fatty alcohols. In the light of previously performed extensive epidemiological studies, the still unclear risk profiles are clarified here of abstainers denying daily alcohol consumption in a reliable manner and those are characterized below as abstainers. Urine pathology was correlated with chronic alcohol consumption before and alcohol-related hypertriglyceridemia with diastolic hypertension using two statistical methods (see www.fida-aha.com). The coded baseline data of three female and three male study groups were evaluated before using two statistical methods each. The previous estimates correlated increase LDL-C (≥150 mg/dl) with increased diastolic blood pressure (90 mmHg, p<0.05) but direct correlations failed between urine pathology and higher age and/or between systolic and/or diastolic hypertension (n.s.). The recognized publications are summarized in the priority document PA10 2019 007 769.5 as state of the art.

An important aspect of the invention comprises then the evaluation of risk factors for protection of the entire endothelium system (see FIG. 2). For the first time, interactions between albumin and HDL-mediated functions are shown here and are combined with a direct protection of cells. Thus, proteophospholiposomes were developed to improve regeneration of the endothelium system among elderly persons as well (≥50 years). The novel FIDA®-algorithms verify the relevance in an objective manner with the novel quotient of HDL to VLDL for the first time and support the relevance with testing of fasting blood glucose. The high variances of circulating albumin limit statistical methods and indicate insufficient standard procedures in regard of albumin. Alternatively, the new FiDA® algorithms verify here the clinical evaluation of hepatorenal risk profiles in an objective manner (Tables 1+2).

The Table 1 shows the novel FiDA®-algorithms. The novel quotients are connected with the testing of fasting blood glucose. The quotients verify the relationships concerning HDL-related protection systems against VLDL-related efflux systems. The protective compartments comprise here the HDL-related influx capacities, for example of cholesterol, of cholesterol esters, normal values of albumin and functional endothelium barriers. The cohort-related normal values are determined here calculating extended standard deviations of abstainers showing then low normal values of fasting blood glucose (FG<100 mg/dl in Table 1-Col. 0 & Table 2-Col. 0, means±1.2×S.D.). Moreover, a normal standard value is calculated here for the first time as VLDL-cholesterol (VLDL-C)=total cholesterol(C)−(LDL-C+HDL-C). The relevant impairments are unveiled with the novel ratio of HDL-C/VLDL-C. The normal FiDA® algorithms verify healthy values of persons recording healthy lifestyle (FIDA®: Alb/Trig≥30, VLDL-C<32 mg/dl, FG<100 mg/dl, HDL-C/VLDL-C>2, Gamma-GT<28 U/l, U-albumin/U-creatinine<30 mg/g). On the other hand, critical FIDA®-formula verify the defects which often overlap with chronic alcohol consumption in an objective manner (FIDA®: Alb/Trig<30, VLDL-C≥32 mg/dl, FG≥100 mg/dl, HDL-C/VLDL-C≤2, GammaGt≥28 U/l, U-Alb/U-Krea≥30 mg/g). At least two critical values of the novel FiDA® algorithms justify a critical forecast. The isolated microalbuminuria is tested with commercially available urine sticks and with standard testing in the laboratory and shows the need of better, more innovative tests to determine emigrated plasma proteins and their proteophospholipids.

The Table 1 of the present invention shows unexpected interrelationships in the context of circulating albumin (P-albumin). Decreased values of P-albumin overlap here with low HDL-C while low HDL-C overlaps with low albumin only among alcohol consumers (Table 1, Col. 1-4). Thus, native albumin of abstainers can protect HDL-particles vice versa e.g. against endothelium-related degradation as explanation of these symptoms. Direct dual effects of albumin are evidenced here showing that too high values of P-albumin overlap with rise of urinary albumin and with diastolic hypertension even among abstainers. Albuminuria often overlaps with critical FiDA® algorithms among chronic alcohol consumers (Table 1, Col. 6).

The classified clinical data allow the conclusions and forecasts with the following definitions. Isolated albuminuria indicates disturbed luminal matrix layers e.g. due to dehydration. Sustainable albuminuria with diastolic hypertension determines albumin emigration and disturbed vasodilation. Elevated fasting blood glucose and critical FiDA® algorithms indicate the hepatic dysregulation of gluconeogenese. On the other hand, standard diagnostic tests of urines show the weakness with the Tables to be then replaced with the coming novel tests and testing procedures of the priority documents. Alternatively, the relevance of albuminuria is determined here with testing blood pressure and with FiDA® algorithms. The standard ratio is used of urinary albumin to urinary creatinine while the tubular reabsorption of urinary albumin cannot be assessed therewith. Indeed, the high variances lead away from mono-factorial evaluations. The quality of urinary albumin is of decisive relevance in respect to a possible fibrotic risk of the tubular system and it is relevant to determine the dual role of albumin as well which can be verified with the novel diagnostic tests in an objective manner. The priority document PA 10 2019007 769.5 specifies a novel test for an improved evaluation of urinary albumin in regard of the below mentioned meningeal albumin. The Table 1 confirms the previously estimated overlap of albuminuria and chronic alcohol consumption.

The Table 2 shows a rather non-classified subgroup of elderly men showing a high proportion of alcohol consumers and the tendency to systolic hypertension combined with albuminuria as compared to the cohort-related normal values of normolipidemic abstainers (see legends of Table 2, Col. 1&2). In turn, subgroups are then characterized having higher age (>50 years) and pathological urine samples. It is shown that this subgroup tends to normal LDL-values and to normal blood pressure despite albuminuria and/or hematuria (Table 2-Col. 6). This subgroup tends to hematuria and is then evaluated as having an age-related impairment of the peri-endothelial matrix materials comprising luminal matrix and basal membranes. Altogether, higher age is evaluated as additive risk factor for declined protective functions of the entire endothelium function especially against elevated LDL-cholesterol and/or against fatty alcohols, chemically alkyl-acyl-sn-glycero-3-phosphocholines (LA-paf, alkyl-GPC). The estimated risk of alcohol-related hyperlipidemia is supported with the (pro-)diabetic risk profile and with critical FiDA® algorithms.

The Table 2 shows for the first time the progression of the hepatorenal problems which begin here with albuminuria and diastolic hypertension indicating impairments of the cAMP-dependent vasodilation and diastolic hypertension changing then over to the manifest hepatorenal hypertension and/or to hepatic diabetes. Primarily healthy men are compared and are characterized as having normal lipid values (Table 2, Col. 1 vs. Col. 2-5). The progression of hepatorenal risk profiles overlaps with the high proportion of alcohol consumers (Table 2, Col. 3-5). The albuminuria with diastolic hypertension indicates an increased vasopermeability (phases I+II, Table 2, Col. 3). The dyslipidemic, pro-diabetic risk profiles show the progression (Phase III, Table 2, Col. 4). Hepatorenal defects are then verified regarding leakage of liver cells (Gamma-GT≥28 U/l or LDH-values below). Overall, a low quotient of HDL/VLDL-C shows a relevant impairment of the influx-efflux-systems, as HDL-C is too low and VLDL-C is too high (Table 2, Col. 5, phase IV). The influx-efflux problems are verified in an objective manner and those are relevant because hepatorenal impairments overlap e.g. with manifest systolic and diastolic hypertension and/or with manifest hepatic diabetes (HbA1c: 8.4±3.2%).

The values of the Tables 1 and 2 are evaluated together. The albuminuria with diastolic hypertension is evaluated as impairments of the luminal matrix. The albuminuria together with diastolic hypertension are evaluated as increased emigration leading to subendothelial irritations. Albuminuria with hematuria are evaluated as impaired regeneration of the endothelial defense systems requiring exclusions of other reasons and being then supplemented with the novel proteophospholiposomes. The innovation comprises the objective evaluation of influx-efflux problems with the novel FIDA® algorithms. The influx-efflux problems are supplemented with the novel proteophospholiposomes, facilitating restrictions by means of nonalcohol liquids which enhance the energy flow replacing then adverse alcohol consumption if possible. The novel proteophospholiposomes serve as reservoir of nutritive compounds whereby especially methionine and cysteine-derivatives attract micromaterials and cofactors, promoting the interstitial energy flow.

The Tables 1+2 together provide evidence of relevant influx-efflux-problems. The HDL-related influx systems are weakened and VLDL-related efflux problems of cholesterol, of cholesterol esters trigger the progredient symptoms. In accordance with the invention, the increased vasopermeability is then treated with the novel proteophospholipids. Pharmaceutical compounds can be completed therewith. FIDA® algorithms evaluate sporadic albuminuria which are frequently found among children and/or among pregnant women while albuminuria can further indicate dehydration or malnutrition. The clinical relevance of risk profiles is evaluated with combinations comprising albuminuria, hypertension and alcohol consumption as risk profile for hepatorenal problems. Elderly persons with albuminuria tend to increased vasopermeability and should avoid shifts of interstitial pH values due to dehydrations which is a special stress for the netlike vessel systems of plexus systems. The novel proteophospholiposomes protect cells and are adapted to the size of endothelial pores (about 70-100 nanometer) to remove the VLDL-particles (70-100 nm) from luminal surfaces (FIG. 2). The protection of the endothelium system is summarized as model (FIG. 2).

### 12. PATHOPHYSIOLOGY AND EXPERIMENTAL MEDICINE

The FIG. 1 shows the protection of intact human cells by means of HDL-proteins with yet unpublished own data. Washed intact human thrombocytes are protected with plasma fractions from healthy volunteers (n=3, means, ±1 S.D.). The plasma fractions were formed prior to the experiments using ultracentrifugation and dialysis in an outsourced laboratory. The HDL-fractions or antibodies against human serum albumin protected intact cells against fatty alcohols e.g. against albumin overloaded with fatty ligands. The FIG. 1 serves as representative model for isotonic liquids which can be used as starting materials, as eluates, as carriers for protection of cells. The FIG. 2 shows fenestrated endothelium barriers as model for labeled proteophospholiposomes (XX) which are reduced during endogenous passages layer by layer to interior HDL-like vesicles (X) for protection of subendothelial cells. The nanometer size of the proteophospholiposomes is adapted also for the diagnostic procedures including diagnostic appliances of labeled proteophospholiposomes and/or of labeled HDL-type vesicles. The labeled proteoliposomes are formed layer by layer with fully specified methods and procedures are not limited therewith.

The FIG. 1 shows that binding of [3H]alkyl-acetyl-sn-glycero-phosphocholines ([3H]PAF) to intact washed human platelets is inhibited with 0.5 mg/ml HDL-apoproteins A in the presence of 0.25% delipidated serum albumin (65 pM, 0.5×10⁷ cells, 20° C., 30 min, pH7.4). The thrombocytes are further preincubated with dialyzed, delipidated plasma proteins (0.36 mg/ml), with purified VLDL-fractions (0.9 mg/ml), with IDL (0.02 mg/ml), with HDL (0.5 mg/ml) or with delipidated HSA (0.25%). The preincubation with HDL-peptides (3 min 37° C.) especially inhibits the binding of [3H]alkyl-PAF. Delipidated human serum albumin shows only a modest inhibition of [3H]PAF (0.5 mg/ml, n=3) while a preincubation with HSA-antibodies prior to the last wash inhibits binding of [3H]Alkyl-PAF providing evidence that albumin adducts are transmembrane transporters (0.25% HSA+Anti HSA 2.5 mg/10⁹ cells, 30 minutes at 20° C.). An additive inhibitory effect of thienodiazepines further shows that the HDL-fractions and/or the HSA-antibodies do not directly inhibit PAF-receptors. Thienodiazepines were established before as specific PAF-receptor antagonists (WEB40 nM). The freshly enriched phosphate buffer serves here as representative system for possible carriers of the liposomes for protection of cells (11.9 mM NaHCO3, 137 mm NaCl, 2.68 mM KCL, 1 mM MgCl2, 0.41 mM NaH2PO4, 0.5 mM dextrose). HEPES (5 mM) is chemically 2-(4-(2-Hydroxyethyl-1-piperazinyl)-ethanol-sulfonic-acid is replaced here by cysteine (30 mg/100 ml) during the preparations of proteophospholiposomes. Antioxidants are added to isotonic liquids completing the redox systems and the broad buffer capacity of albumin with the vitamins A, B, C, D, E, whereby vitamin mixtures for cells in culture serve as a representative model. Intact cells are thus specifically inhibited with HDL-fractions or with HSA-antibodies added prior to the last wash. The radioligand assays are started with addition of control cells compared to addition of pretreated cells and the assays are stopped by vacuum filtration. The pharmacological rules allow the conclusion that HDL-proteins and albumin protect cells by means of separate pathways so as the clinical synergistic effects are supported with experiments as well.

Equivalent methods are used excluding negative effects of thioether-acyl-2-acyl-glycerophosphatidylcholines testing and excluding interaction with PAF receptors. Thio-acyl-glycero-phosphatidylcholines (500 nM Thio-GPC) do not at all change the total binding of [3H]PAF. Washed thrombocytes are tested in the presence of thioether-2-acyl-glycero-phosphatidylcholines (500 nM) compared to controls. Only unlabeled PAF shows a competitive inhibition of total [3H]PAF binding (from 22±3 fmol to 6±2 fmol per 10⁸ cells/ml, 500 nM PAF, n=3).

Further experiments show that the buffer capacity of albumin protects intact human thrombocytes using LDH-Kits (Boehringer, Deutschland). Leakage of cells was found with loss of lactate-dehydrogenase (LDH) from cells and increase of LDH was paralleled along duration of high pH values (pH9.5). The normal loss of cellular LDH is about 7±3% in the presence of physiological pH-values (pH 7.4, 0.25% BSA, n=3) and increases to 38±4% in an albumin-free buffer at pH 9.5. On the other hand, 0.25% BSA protects cells as the cellular LDH-leakage decreased to 14% (pH9.5). The broad buffering capacity is evidenced therewith and also the additive synergistic effects of apoproteins A and serum albumin.

Altogether, the experimental data support the additive protection of intact cells by means of HDL-apoproteins A and/or with serum albumin. The additive effects show distinguished allosteric pathways such as antioxidation potency and/or e.g. electrostatic capacities which are antagonized here with HDL-proteins for cell protection against alcohol metabolites mediating influx-efflux-problems (FIG. 1 and Tables 1+2).

A further embodiment of the invention discloses the response to the open questions concerning the significant rise of lysopaf in cerebrospinal fluids during psychotic symptoms compared to persons without cerebral symptoms (see e.g. Ruth-Maria Korth U.S. Pat. No. 5,605,927, Publ. Feb. 25, 1997). In yet unpublished statistical data, a significant increase is verified of lysopaf per 500 μl CSF during inflammatory cerebral disorders compared to paranoid symptoms (p<0.039) or to neurodegenerative disorders (p<0.017) or compared to liquor without any symptoms (p<0.02). Lysopaf significantly increases per 500 μl CSF during psychotic symptoms compared to paranoid delusions (p<0.03) or compared to neurodegenerative disorders (p<0.005) while no significancy was reached comparing psychotic symptoms to inflammatory cerebral disorders. Albumin is verified here for the first time as acceptor e.g. of lysopaf and the cerebral formation of lysopaf is unveiled for the first time with yet unpublished statistical data. A significant rise of albumin in cerebrospinal fluids (CSF) was not directly related e.g. during psychotic symptoms while psychotic symptoms were directly correlated with increased levels of lysopaf in cerebrospinal fluids. The disclosed innovation provides intermediate layers comprising nascent albumin as complementary mengingeal acceptors which relief cerebral transporters by means of extracerebral lymphomeningeal systems (CSF). Composites with cysteine and/or stimulating procedures could enhance here uptake considering that only 1% of plasmatic albumin is taken up by the extracerebral meningeal drainage systems. The novel tests of the priority document are needed as lysopaf is a stable alcohol metabolite, chemically 1-O-alkyl-lyso-glycerophosphocholines which is significantly higher during psychotic symptoms and is further correlated here with inflammatory cerebral disorders (7±2 ng lysopaf per 500 μl CSF) compared to values in the absence of symptoms (3+1 ng lysopaf per 500 μl CSF). Lysopaf is a stable metabolite and lysopaf is formed in the central nervous system also during inflammatory disorder as well e.g due to transformation of cerebral plasmalogenes and/or upon activation of cerebral macrophages. Continued testing procedures require practical testing procedures which are overdue because lysopaf is found in plasma in a detectable concentration area as well (0.2±0.2 ng/mg lysopaf per mg plasma proteins). A further reason is provided disclosing the tests in the priority documents.

The FIG. 2 shows the endothelial system as model. The entire endothelium system is shown here as a model for diagnostic appliances of the labeled proteophospholiposomes in accordance with the invention. The entire endothelium system comprises the endothelium layer (A), the subendothelial cells (B: pericytes, smooth muscle cells, macrophages), the matrix materials, the luminal layers and the basal membranes (C). Especially the fenestrated endothelium barriers are luminally protected here with proteophospholiposomes (XX) (e.g. of glomeruli, of glands, of some plexus capillaries). The plasma proteins are albumin (D: 20 nm) and HDL which correspond to the size of endothelial pores (20-70 nm) and which are smaller than VLDL-particles (E: about 70-100 nm) containing apoproteins B and E (apoB+E). First, native HDL contains only Apoproteins A1, A2 and lecithins integrating then cholesterol ester and/or apoproteins E. The novel multilamellar proteophospholiposomes (XX: 70-100 nm) reduce themselves during passages to the interior HDL-like vesicles (X) which remain protected with thio-acyl-phosphatidylcholines reaching therewith the perivascular, interstitial spaces, which take over stored materials and/or cleave materials in a thioclastic manner (YZ). The plasmatic VLDL-particles or the HDL-particles are the known plasmatic acceptors of cholesterol esters. Impaired efflux e.g. during oxidative stress and/or inflammatory reactions precipitations or storage of aggregates is enhanced. The hyperphosphorylated or misfolded proteins (e.g. β-amyloids or tau-proteins) correspond here with the stable alcohol metabolite lysopaf. The innovation is disclosed with the broad buffer capacity and with the ionic micromaterials promoting the cerebral, the interstitial energy flow.

Legend of FIG. 1: HDL fractions protect intact washed human thrombocytes in an enriched phosphate buffer. Details can be found in chapter 12.

Legend of FIG. 2: The endothelium system (A) provides the subendothelial cells (B), the peri-endothelial matrix materials (C), reflection of anionic albumin (D) and VLDL-related efflux systems (E). The proteophospholiposomes (XX) reduce their size during passages and become HDL-type vesicles (X) protecting cells and cleaving metabolites of fatty alcohols. Details can be found in chapter 12.

TABLE 1
Normal values (0: means ± 1.2 × S.D.) of functional protectors, namely
the albumin group and the endothelium groups are compared. Abstainers (col. 1,
3, 5) show healthy values versus cohorts with frequent alcohol problems (AHA: 2, 4, 6)

| Characteristics | 0: Healthy Data | 1: HDL-C < 45 | 2: HDL-C < 45 | 3: S-Albumin < 4 |
|---|---|---|---|---|
| means ±1/±1.2 × SD | Nonalcohol, n = 51 | Nonalcohol | 53% AHA | Nonalcohol |
| HDL-Cholesterol | 57 ± 16 > 38 | select. 39 ± 3 | select. 39 ± 12, n | 36 ± 2 ≤ 38 |
| LDL-Cholesterol | 128 ± 36 < 154 | 118 ± 42 | 142 ± 67 | 113 ± 32 |
| HDL-C/LDL-C | 2.5 ± 1.1 < 3.8 | 3.6 ± 0.7 | 4 ± 2 | 3.0 ± 0.5 |
| Serum(S)Albumin g/dl | 4.7 ± 0.6 > 4 | 4.9 ± 1 | 4.0 ± 0.7 | select 3.3 ± 0.8 |
| Alb/Trig | 54 ± 19 ≥ 30 | 46 ± 16 | 24 ± 11 | 37 ± 4 |
| Total Cholesterol | 188 ± 37 | 175 ± 31 | 223 ± 87 | 171 ± 30 |
| age, years | 36 ± 5, n = 51 | 32 ± 1 | 39 ± 12 | 34 ± 10 |
| age > 50 in % | 51 of 131, 39% | 8 of 131, 6% | 13 of 131, 10% | 6 of 131 |
| BMI kg/m2 | 26 ± 4 < 29 | 23 ± 2 | 28 ± 6 | 23 ± 2 |
| Triglycerides mg/dl | 92 ± 35 < 134 | 129 ± 48 | 196 ± 87 | 105 ± 14 |
| U-Albumin/U-Kreatinin | 19 ± 10 | 24 ± 2.3 | 21 ± 4 | 23 ± 2.8 |
| Fasting glucose(FG)mg/dl ″ | 85 ± 10 > 73 ≤ 97 | 85 ± 4 | 103 ± 38 | 83 ± 4 |
| Hepatic GGT U/L | 16 ± 10 < 28 | 17 ± 5 | 27 ± 17 | 19 ± 4 |
| VLDL-C mg/dl | 17 ± 12 < 32 | 19 ± 6 | 30 ± 21 | 18 ± 10 |
| HDL/VLDL-C | 9 ± 2 > 6.6 | HDL/VLDL > 2 | 0.96 ± 0.6 | 5.4 ± 6.5 |
| CRP mg/dl | 0.4 ± 0.1 < 0.5 | 0.4 ± 0.2 | 0.4 ± 0.2 | 0.3 ± 0 |
| P-creatinine mg/dl | 0.9 ± 0.2 < 1 | 0.86 ± 0.1 | 1 ± 0.2 | 0.9 ± 0.1 |
| AlKohol use, AHA % | elect. non | elect. Non | 71% of 19 | elect. non |
| Smoking % | elect. non | 7 of 8, 88% | 65% of 19 | elect. non |
| U-albumin mg/l | 23 ± 4 < 28 | 22 ± 10 | 38 ± 23 | 28 ± 3 |
| Syst. RR mmHg | 123 ± 16 < 140 | 125 ± 12 | 125 ± 12 | 126 ± 13 |
| Diast. RR ″ | 82 ± 9 ≤ 90 | 85 ± 4 | 84 ± 4 | 82 ± 3 |
| % Urine Pathology | 14% of 51 | sporadic | 58% of 19 | sporadic |

| Characteristics | 4: P-Albumin < 4 | 5: P-Albumin > 5 | 6: P-Albumin > 5 |
|---|---|---|---|
| means ±1/±1.2 × SD | 60% AHA | Nonalcohol | 100% AHA |
| HDL-Cholesterol | 42 ± 8 | 57 ± 21 | 53 ± 5 |
| LDL-Cholesterol | 143 ± 80 | 115 ± 49 | 162 ± 17 |
| HDL-C/LDL-C | 3.6 ± 2 | 2.4 ± 1.2 | 2.8 ± 1.2 |
| Serum(S)Albumin g/dl | elect. 3.6 ± 0.5 | el. 5.93 ± 0.6 | el. 7.0 ± 2.1 > 5 |
| Alb/Trig | 20 ± 5 | 61 ± 11 | 37 ± 13 |
| Total Cholesterol | 143 ± 80 | 189 ± 58 | 259 ± 47 |
| age, years | 44 ± 13 | 32 ± 11 | 44 ± 10 |
| age > 50 in % | 10 of 131, 8% | 6 of 131, 5% | 5 of 131, 45 |
| BMI kg/m2 | 25 ± 6 | 23 ± 5 | 29 ± 5 |
| Triglycerides mg/dl | 209 ± 239 | 102 ± 22 | 417 ± 273 |
| U-Albumin/U-Kreatinin | not found | not found | 44 ± 10 |
| Fasting glucose(FG)mg/dl ″ | 86 ± 10 | 85 ± 6 | 118 ± 29 > 100 |
| Hepatic GGT U/L | 66 ± 117 | 13 ± 4 | 162 ± 147 |
| VLDL-C mg/dl | 31 ± 15 | 14 ± 9 | 57 ± 48 |
| HDL/VLDL-C | 1.8 ± 1.3 | 6.3 ± 4.7 | 5.3 ± 4.6 |
| CRP mg/dl | 0.3 ± 0.1 | 0.5 + 0.2 | 0.6 ± 0.3 |
| P-creatinine mg/dl | 0.83 ± 0.14 | 0.99 + 0.1 | 0.9 ± 0.3 |
| AlKohol use, AHA % | 60% of 10 | elect non | el. 87 ± 42 g |
| Smoking % | 70% of 10 | elect non | 67% of 6 |
| U-albumin mg/l | 38 ± 16 | 30 ± 16 | 32 ± 17 |
| Syst. RR mmHg | 126 ± 16 | 131 ± 19 | 139 ± 12 |
| Diast. RR ″ | 83 ± 8 | 90 ± 6 | 94 ± 16 > 90 |
| % Urine Pathology | 60% of 10 | 67% of 6 | 40% of 5 |

Col 0: Healthy, coded values of healthy abstainers at baseline (FIDA ®: Alb/Trig ≥ 30, VLDL-C < 32, FG < 100 mg/dl, GGT ≤ 28 U/l).
Col1 + 3: Low values of S-albumin and of HDL-C of abstainers overlap in the absence of albuminuria (U-albumin/U-crea: ≤30 mg/g).
Col. 2 + 4: Dysalbuminemia overlaps with alcohol-related rise of vasopermeability, which is corelated here with albuminuria (p < 0.05).
Col. 5 + 6: High S-Albumin values overlap with urine pathology and diastolic hypertension with or without alcohol consumption but Col. 6: (pro)diabetic, hepatorenal risk profiles are unveiled (FIDA ®: VLDL-C ≥ 32, FG > 100 mg/dl, GGT ≥ 28 U/l, HDL/VLDL-C ≤ 2, HDL/VLDL < 2, U-alb/U-crea ≥ 30 mg/g)
Conclusion: Serum-albumin, HDL-C and endothelium barriers are interrelated protectors.
Innovation: Apoproteins A are combined for cell protection when high albumin is an endogenous hypertensive risk factor.

TABLE 2
Classification of men's coded biomarkers with normal values (col. 1), age (≥50 years, col. 2), diastolic
hypertension (col. 3), dyslipidemia (col. 4), hepatic defects (col. 5), age + albuminuria (col. 6).

| | Norm. | | 3)Diast. | 4)Dyslipid. | | 6)Age + |
|---|---|---|---|---|---|---|
| Targets | Triglycerides | 2)Age | Hypertension | phaseIII | 5)Hepat-phaseIV | Albuminuria |
| n of 131 | n = 41 | 11 of 71 | n = 38 | 17 of 58 | n = 13 | 12 of 131 > 45 |
| Age, years | 35 ± 8 | select. 58 ± 7 | 39 ± 11 | 46 ± 13 | 45 ± 12 | select 53 ± 5 |
| VLDL-C mg/dl | 14 ± 8 < 32 | 40 ± 22 | 24 ± 19 | select. 51 ± 21 ≥ 32 | 40 ± 18 > 32 | 27 ± 19 < 32 |
| Cholesterol mg/dl | 196 ± 54 | 238 ± 68 | 191 ± 50 | 260 ± 53 | 190 ± 58 | 210 ± 37 |
| LDL-C ″ | 130 ± 40 | 160 ± 70 | 143 ± 48 | 175 ± 60 | 120 ± 84 | 124 ± 55 |
| HDL-C ″ | 52 ± 12 > 38 | 52 ± 14 | 55 ± 21 | 46 ± 10 | 38 ± 8 | 55 ± 29 |
| LDL/HDL | 2.4 ± 0.9 < 3.8 | 3.0 ± 0.7 | 2.9 ± 1.5 | 3.6 ± 1.8 | 3.1 ± 0.8 | 3 ± 1.6 |
| HDL/VLDL-C | 11 ± 21 > 2 | 4.8 ± 2 | 8 ± 11 | 1.1 ± 0.4 < 2 | 0.95 ± <2 | 2.9 ± 1.8 > 2 |
| Non-HDL-C mg/dl | 140 ± 47 < 197 | 164 ± 56 | 157 ± 51 | 215 ± 55 > 197 | 152 ± 52 | 205 ± 27 |
| Triglycerides mg/dl | select 113 ± 58 < 134 | 198 ± 100 | 172 ± 133 | 239 ± 179 > 134 | 200 ± 79 | 216 ± 23 |
| Serum-Albumin g/dl | 4.7 ± 0.6 | 4.1 ± 0.9 | 4.8 ± 0.8 | 4.7 ± 1.5 | 5.1 ± 2.3 ≥ 5 | 4.9 ± 0.2 |
| Alb/Trig | 53 ± 22 > 30 | 37 ± 13 | 40 ± 17 | 35 ± 23 | 31 ± 15 | 37 ± 13 |
| Fasting glucose mg/cl | 86 ± 10 < 100 | 98 ± 30 | 96 ± 21 | 108 ± 31 > 100 | select 133 ± 20 | 92 ± 13 |
| Hepatic GGT U/l | 24 ± 10 < 28 | 25 ± 18 | 48 ± 53 | 107 ± 127 | 85 ± 106 > 28 | 24 ± 10 |
| Alcohol g/Tag | 22% of 41 | 55% of 71 | 66% of 38 | 82%: 100 ± 27 g | 69% von 13 | 25% von |
| Smoking Zig./Tag | 22% of 41 | 27% of 71 | 37% of 38 | 59%: 24 ± 10cig | 38% von 13 | 25% von 12 |
| Systol. RR mmHg | 125 ± 15 < 140 | 140 ± 19* | 130 ± 16 | 134 ± 13 | 144 ± 15 > 140 | 128 ± 13 |
| Diast. RR mmHg | 87 ± 5 < 90 | 87 ± 17 | select: 91 ± 10* | 85 ± 9 | 92 ± 10 > 90* | 88 ± 10 ≤ 90 |
| BMI kg/m2 | 27 ± 2 | 28 ± 5 | 28 ± 5 | 30 ± 6 | 30 ± 6 | 28 ± 5 |
| P-creatine mg/dl | 0.9 ± 0.1 < 1.1 | 0.9 ± 0.3 | 1.03 ± 0.3 | 0.9 ± 0.2 | 0.8 ± 0.1 | 1 ± 0.4 |
| Uric acid mg/dl | 6.2 ± 1.2 | 5.3 ± 2 | 5.4 ± 2.1 | 5.2 ± 2 | 5.8 ± 2 | 4.4 ± 1.3 |
| Albuminuria mg/l | 26% 28-30 mg/l | 55%: 37 ± 27 | elect. 32 ± 15 ≥ 30 | 53%: 37 ± 10 mg/l | 62%: 29 ± 10 | 11 of 12 > 30 mg/l |
| Hematuria | non | n = 4 | n = 4 | n = 1 | n = 1 | n = 5 |

Col. 1: Normal triglyceride values overlap with normal FIDA ®formula (Alb/Trig ≥ 30, VLDL-C < 32, FG < 100 mg/dl, GGT < 28 U/l).
Col. 2: Age often overlaps with alcohol problems, correlated with urine pathology (p = 0.04), not directly with hyperetnsion(p > 0.2).
Col. 3: Albuminuria (phaseI) with diastolic hypertension (phase II) overlap with chronic alcohol consumption (30 ± 9 g/day).
Col. 4: Dyslipidemia with high VLDL-C, HDL/VLDL-C < 2 objectively unveil unhealthy alcohol consumption with FiDA ®formula.
Col. 5: Manifest hepatic defects, high VLDL-C, diabetes (HBA1c > 6.5%) and sustainable hypertension verify unhealthy alcohol use.
Col. 6: Albuminuria and age are not directly correlated and barrier problems are neverthelss shown especially during hematuria.
Conclusion: Alcohol-related defects are progredient and are objectively unveiled with FIDA ®formula.
Innovation: Proteophospholiposomes protect membranes against alcohol metabolites, against alkyl-acyl-GPC (see FIG. 1).

1) Proteophospholiposomes containing inner HDL-like vesicles characterized by a composite comprising apoproteins A with at least one polypeptide, selected from the group of the albumins, the transthyretin-prealbumins and with at least one cysteine group, wherein the anionic polypeptide composite is coated with at least one layer containing acyl-phosphatidylcholines and at least one cysteine group for stabilization and for enrichment of ionic micromaterials and at least one cofactor for application forms to protect cells, for health promoting appliances for diagnostic appliances with at least one labeled cofactor.

2) The proteophospholiposomes according to claim 1, selecting apoproteins A from the group of apoproteins A1, A2, E2 and composed by means of cysteine groups and containing at least one polypeptide selected from the group of albumins comprising serum albumin, lactalbumin, Vitamin D binding proteins, transthyretin-prealbumins and wherein the acyl-phosphatidylcholines are selected from the group of zwitterionic acyl-phosphatidylcholines and wherein the cysteine groups are selected for stabilization from the group of thio-phosphatidylcholines and at least one cofactor is selected for dietary, dermal, transdermal, cosmetic, oral, pharyngeal, nasal, lacrimal, pulmonal or epimeningeal application forms, which are prepared for health promotion, for diagnostic appliances.

3) The proteophospholiposomes according to claim 2 for dietary appliances, wherein starting materials are selected from milk products and certified organic oils which are enriched with purified, synthetic or semisynthetic polypeptides and which bind zwitterionic acyl-phosphatidylcholines by means of anionic capacities for oral intestinal application forms to reach at least one plexus system for care of mouth, intestine, teeth, the intestine for neuro vegetative, cholinergic equilibration, for promotion of lymphatic interstitial energy flow, for protection of cells, for equilibration of influx-efflux problems especially during an elevated vasopermeability, in the presence of critical FiDA® algorithms.

4) The proteophospholiposomes according to claim 2 for dermal, transdermal, cosmetic appliances, wherein the inner HDL-like vesicles are protected in a coated manner with certified organic acyl-phosphatidylcholines containing at least one esterified fatty acid from the group of stearic fatty acids, of oleic fatty acids, of linoleic, of linolenic fatty acids as vitamin F, wherein at least one phospholipid layer contains thio-phosphatidylcholines attracting ionic microelements, cofactors and wherein at least one intermediate layer contains albumin as reservoir for natural moisturizing factors which are obtainable from plant products, algae products, honey products, plankton products, milk products, fish products and which contain acetyl coenzymes A and wherein the vesicles are coated with at least one further external layer containing fats, lipids and wherein the cofactors are selected from the group of retinols, cholecalciferoles, tocopherols, the Ginkgoloides, the xanthines which can be enriched to pass intercorneal lipid layers and for promotion of the netlike microcirculation of the hypodermis.

5) The proteophospholiposomes according to claim 2 for pulmonal appliances containing synthetic, semisynthetic, purified proteophospholipids with synthetic dipalmitoyl-phosphatidylcholines and which can be prepared with sterile isotonic solutions, with recombinant peptides, with human albumin for direct installations, for bronchial installations, subcutaneous, intravenous, plasmapheretic application forms or for pharyngeal, nasal, dermal, inhalative application forms containing dipalmitoyl-phosphatidylcholines wherein the surfactant phospholipids are enriched to stabilize alveoli, to equilibrate cholinergic binding sites of the pulmonal plexus and of pulmonal macrophages and to protect pulmonal capillaries, wherein one or more layers are prepared with albumin, cysteine, with acetylcysteines to activate thioclastic, xenobiotic, mucolytic systems, for cleavage of peroxides in the presence of sick neighboring tissues and as reservoir of ionic micromaterials, of antioxidants and with a broad buffering capacity of albumin.

6) The proteophospholiposomes according to claim 2 for cerebral appliances, containing the inner HDL-type vesicles as a composite comprising apoproteins A with transthyretin-prealbumins and cysteines and coated with at least one surrounding layer containing synthetic or semisynthetic dipalmitoyl-phosphatidylcholines with thio-phosphatidylcholines and ionic micromaterials and containing at least one cofactor from the group of vitamins, of vitamins A, B, D, E, F, from the group of cholinergic agonists, the apoproteins E2, the reelin group and wherein the vesicles are further coated with one or more layers for epimeningeal, lacrimal, nasolacrimal, nasopharyngeal, retropharyngeal, buccal, lingual, dermal, parenteral application forms to balance extracerebral and cerebral plexus systems, for promotion of lymphomeningeal drainage systems and of influx-efflux systems, of interstitial energy flow, of ionic pulsations, of cholinergic systems, wherein at least one layer contains albumin as reservoir comprising nutritive components, for a broad buffering capacity, for extracerebral, meningolymphatic drainage systems, for equilibration of bidirectional transport systems of the Chorioid Plexus, for cleavage of useless metabolites and for inhibition of re-absorption by means of transthyretin-prealbumins.

7) Proteophospholiposomes according to claim 1 for diagnostic appliances, wherein labeled cofactors promote the electromagnetic capacities of the proteophospholipids and are prepared as test devices or that labeled cofactors are included which use symmetrical or asymmetrical charges for preparations of and with labeled proteophospholiposomes, wherein especially the cysteine groups of albumin, the thiolgroups are labeled for tests, for testing procedures, for diagnosis of matrix materials, for presentation of an interstitial energy flow, for electromagnetic measurements, for imaging procedures without irritation of the endothelium system as a whole.

8) The proteophospholiposomes with the diagnostic appliances in accordance with claim 7, wherein the cysteine groups of polypeptides are provided as thiol-groups with labeled fusion partners, wherein electromagnetic markers, spin labeled markers are selected for diagnostic procedures, for imaging procedures for diagnosis of the entire endothelium system, comprising matrix materials, endothelium barriers, interstitial cells and for presentation of an interstitial energy flow without irritations.

9) The proteophospholiposomes with the components and appliances in accordance with one of the claims 1 to 6, wherein the cysteine groups are prepared as essential methionine derivatives, wherein milk products, nuts, seedlings can serve as starting materials which are enriched in stationary media to form proteophospholiposomes further comprising the cytidine phosphates, acetyl Coenzymes A and choline groups diffusing as cholinergic, ionic micromaterials and wherein the apoproteins A are prepared with albumin and electrolytes corresponding to an experimental model for protection of cells and wherein the inner HDL-like vesicles are prepared containing one or more layers with exterior membranes, coatings or capsules adapted for application forms, to protect cells, for a neuro vegetative balance of plexus systems and for equilibration of influx-efflux-systems with cAMP-agonists especially during enhanced vasopermeability.

10) The proteophospholiposomes with the appliances according to claim 1 wherein cysteine groups stabilize the binding of ionic micromaterials and of antioxidants and wherein the layers are buffered and titrated in consideration of the endogenous targets, wherein the lipid to protein quotient is equilibrated for plasmatic-lymphatic, epimeningeal application forms and wherein an intermediate layer with enriched albumin supports the anionic corneal skin barriers and serves as reservoir of antioxidants, electrolytes, peptides, amino acids, moisturizing factors for protection of skin cells, for a broad buffering capacities, for regeneration of netlike vessels of the hypodermis and for cleavage of useless materials by means of thioclastic thiogroups.

11) Preparation for manufacturing the proteophospholiposomes in accordance with claim 10, wherein the proteophospholiposomes are prepared, enriched, buffered, titrated layer by layer (20° C.), wherein a dispersion is initially prepared which is alternately incubated with water in oil and oil in water situations so as vesicles (a), micelles (b) and then liposomes (c) form themselves and wherein the preparations can be repeated for preparation of multilamellar Proteophospholiposomes (d) and wherein particles arrange themselves by means of ultrasound pulses at cool temperatures (about 4° C.) and wherein the particles (a, b, c, d) can be enriched alone and/or together by means of ion exchange procedures and the stationary media can be optionally used as carriers or are wasted as supernatant during the preparation of dietary, dermal, transdermal, oral, lymphatic, dermal, pulmonal, inhalative, pharyngeal, nasolacrimal application forms and appliances are enforced or the electromagnetic capacities of proteophospholipids are enforced or labeled for diagnostic appliances.

12) The proteophospholiposomes with the manufacturing procedure of claim 11, wherein milk serves as watery stationary medium, wherein certified organic milk products are enriched with apoproteins A, wherein selected milk products contain cysteines (about 30 mg/100 g), antioxidative vitamins (A, B, C, D, E, F) and minerals and their amounts are adapted to the daily need and wherein large droplets of milk are initially enriched and their sizes are then reduced by means of ultrasound pulses (4° C.).

13) The proteophospholiposomes with manufacturing procedures of claim 11 wherein sterile isotonic liquids are prepared for parenteral or direct installations containing at least 0.5 mg/ml apoproteins A with 0.25% delipidated serum albumin and at least 11.9 mM NaHCO3; 137 mM NaCl, 2.68 mM KCL, 1 mM MgCl2, 0.41 mM NaH2PO4, 0.5 mM Dextrose, wherein HEPES-sulfat groups (5 mM) are replaced by cysteine (30 mg/100 ml) and wherein acyl-phosphatidylcholines are added (500 nM) and wherein the peptide composite is selected from the group of purified sterile plasma fractions or of recombinants.

14) The manufacturing procedure of one of the claim 12 or 13, wherein the polypeptide composite originates from dispersions, wherein HDL-like vesicles are initially formed (a) by means of ultrasound pulses (4° C.) and these vesicles have cationic surfaces which can be enriched in a reflected manner by means of outsourced cationic surfaces forming then micelles (b) in a repeated watery situation and those have then anionic surfaces and can be enriched in a reflected manner by means of an outsourced matrix-like, anionic surfaces and form then liposomes (c) in a neutrally charged lipid medium and proteophospholiposomes (d), wherein vesicles (a), micelles (b), liposomes (c), proteophospholiposomes (d) are composed alone and/or together using eluates.

15) The proteophospholiposomes in accordance with claim 11 or 14, wherein the vesicles (a), micelles (b), liposomes (c), proteophospholiposomes (d) are enriched alone or together by means of reflections and are composed with eluates and are then prepared, offered, applied as microsomes alone or together in an outwardly uniformed form for cosmetic, dermal, dietary compounds or as remedies protected with outer coatings, with capsules.
